(19) United States
(12) Patent Application Publication
(10) Pub. No.: US 2012/0035418 A1
(43) Pub. Date: Feb. 9, 2012

(54) IMAGING SENSOR WITH THERMAL PAD FOR USE IN A SURGICAL APPLICATION

(76) Inventors: Joshua D. Talbert, Cottonwood Heights, UT (US); Jeremiah D. Henley, Cottonwood Heights, UT (US); Donald M. Wichern, South Ogden, UT (US)

(21) Appl. No.: 13/024,266

(22) Filed: Feb. 9, 2011

Related U.S. Application Data
(60) Provisional application No. 61/302,811, filed on Feb. 9, 2010.

(51) Int. Cl. A61B 1/04

(52) U.S. Cl. 600/109

(57) ABSTRACT

A system, apparatus and methods for providing a single use imaging device having thermal and electrical safeguards for sterile environments is disclosed and described. A single use high definition camera used for general purpose surgical procedures including, but not limited to: arthroscopic, laparoscopic, gynecologic, and urologic procedures, may comprise an imaging device that is a sterile and designed to ensure single use. The imaging device may further include a thermal pad that is thermally conductive, but relatively electrically insulating.

### CROSS-REFERENCE TO RELATED APPLICATIONS

This application claims priority to U.S. Provisional Application No. 61/302,811 filed on Feb. 9, 2010, which is titled IMAGING SENSOR WITH THERMAL PAD FOR USE IN A SURGICAL APPLICATION.

### STATEMENT REGARDING FEDERALLY SPONSORED RESEARCH OR DEVELOPMENT

Not Applicable.

### BACKGROUND

The disclosure relates generally to imaging devices used during surgical procedures to visualize a surgical area, and more particularly, but not necessarily entirely, to an imaging device and imaging sensor comprising a thermal pad used to dissipate and direct heat from the imaging sensor.

Endoscopic surgery is experiencing rapid growth in the medical field. Endoscopy is a minimally invasive surgical procedure that is used to analyze the interior of a body cavity or interior surfaces of an organ by inserting a tubular member into the body cavity through a minor or minimal incision. A conventional endoscope is generally an instrument with a light source and an image sensor or device for visualizing the interior a body cavity. A wide range of applications have been developed for the general field of endoscopes including, but not necessarily limited to: arthroscope, angioscope, bronchoscope, choledochoscope, colonoscope, cytoscope, duodenoscope, enteroscope, esophagogastro-duodenoscope (gastroscope), laparoscope, laryngoscope, nasopharyngo-neproscope, sigmoidoscope, thoracoscope, and utererscope (hereinafter referred to generally as “endoscope”). The advantages of endoscopy include smaller surgical incisions and less soft tissue damage. As a result, there is significantly less discomfort and pain for the patient as well as a decrease in recovery time.

The advantages of minimally invasive surgery performed with the help of an endoscope are well known and understood in the medical field. As a result, there have been a growing number of devices for use with endoscopes for delivering, for example, diagnostic, monitoring, treatment, operating instruments, tools, and accessories (collectively, “tools”) into the observation field and working space of the physician's endoscope.

As part of forming an image of the surgical site, the endoscope includes a light source and an image sensor. Endoscopes may also incorporate more than one tubular member for observation or operation within the body, such as a working channel for passing diagnostic, monitoring, treatment, or surgical tools through the endoscope. Endoscopes include glass lenses and an adjustable ocular or eye piece, a lateral connection for a light conductor, an adaptor that allows focusing, and a camera head. This configuration is also called a video endoscope.

Additionally, imaging devices are subject to governmental regulations, for example the FDA in the United States, to protect patients and surgeons from potential burns and electric shock that may result in injury. These devices may be made in accordance and consistent with International Electrotechnical Commission (“IEC”) standard 60601-1. The disclosure is directed to an apparatus, assembly and method for removing heat from a medical device, specifically an imaging device such as a camera head, while isolating the patient from the heat source.

It is axiomatic that strict sterilization of the operating room and surgical equipment is required during any surgery. The strict hygiene and sterilization conditions required in a “surgical theater,” i.e., operating or treatment room, necessitate the highest possible sterility of all medical devices and equipment. Part of that sterilization process is the need to sterilize anything that comes in contact with the patient or penetrates the sterile field, including the endoscope and its attachments and components. It will be appreciated that the sterile field may be considered a specified area, such as within a tray or on a sterile towel, that is considered free of microorganisms; or the sterile field may be considered an area immediately around a patient that has been prepared for a surgical procedure. The sterile field may include the scrubbed team members, who are properly attired, and all furniture and fixtures in the area.

In recent years there has been a trend of providing a single use endoscope and components as a packaged, sterilized product, similar to a package containing a surgical implant, such as a knee or hip implant. In terms of endoscopy, instead of using endoscopes that have been reconditioned for each new surgery through traditional sterilization procedures, it means using a single use endoscope and components that are delivered to the hospital in a sterilized package. Due to this trend, it has become increasingly difficult to ensure that each endoscope and its components are properly cared for, used and sterilized for single use and not simply re-sterilized using traditional sterilization procedures.

Traditional drawbacks or problems of video endoscopes include a lack of image quality, the need for sterilization and high manufacturing cost as well as high processing cost. To address these and potentially other problems, the disclosure utilizes unique imaging devices or sensors in addition to a unique method, system and process for providing and reclaiming single use imaging devices.

The features and advantages of the disclosure will be set forth in the description that follows, and in part will be apparent from the description, or may be learned by the practice of the disclosure without undue experimentation. The features and advantages of the disclosure may be realized and obtained by means of the instruments and combinations particularly pointed out herein.

### SUMMARY OF THE DISCLOSURE

An embodiment may comprise a single use camera used for general purpose surgical procedures including, but not limited to: arthroscopic, laparoscopic, gynecologic, and urologic. An embodiment may comprise an imaging device that is a sterile and designed to ensure single use. An embodiment may be an imaging device that comprises a single imaging sensor, either CCD (charge coupled device) or CMOS (complementary metal oxide semiconductor), encased in a molded plastic housing. The imaging device may further comprise the means to be attached to an optical coupling device, using C-Mount and CS-Mount threads or another proprietary or unique connection method. It is within the disclosure to include integrated optical systems, such that no specific coupling means is required. The imaging device may further comprise a cable or wireless method to transmit data to and from a camera control unit. An embodiment may further comprise a thermal energy dissipation means such as a heat sink or cooling mechanism.

An embodiment may comprise a thermal pad that may be substantially rigid or may be deformable. An embodiment may comprise a thermal pad that may be configured to cover substantially all of the surface contact area between the heat sink and any heat generating circuitry. An embodiment may comprise a thermal pad that may be configured to cover a portion of the surface contact area between the heat sink and any heat generating circuitry. An embodiment may comprise a thermal pad that may be configured to cover a plurality of surface contact areas. An embodiment may comprise a thermal pad that may comprise a plurality of thermal pads working on a single surface contact area. An embodiment may comprise a plurality of thermal pads working on a plurality of surface contact areas. An embodiment may comprise a thermal pad having areas of varying thickness configured to accommodate the structure and geometry of surrounding components. An embodiment may comprise a thermal pad comprising a plurality of materials. An embodiment may comprise a thermal pad comprising fold lines.

In an embodiment, information will be recorded in the memory of the imaging device each time it is used in a procedure or quality control (QC) checked at the manufacturer. This information may be used to evaluate usage time, expiration date, etc. An embodiment may comprise features to ensure that the imaging device is only used once and that the imaging device is safe for use.

In an embodiment, the imaging device may be fully covered in plastic having a sensor heat sink to ensure the camera head meets cardiac floating (CF) and body floating (BF) ISO standards. An embodiment may comprise an imaging device that may be stamped with the current time when plugged into a console in the field after a quality control check has been performed. This time may be used as a baseline for usage. If the imaging device is powered off for a predetermined period of time, which may be equivalent to a sterilization cycle, then the imaging device will not function. The imaging device may display an onscreen message telling the user that the camera has already been used and will not allow current operation. These features ensure the imaging device will not be used more than one time per sterilization cycle and further ensures that proper sterilization is performed by the manufacturer or other authorized source. This function is to protect the patient and the doctor from an invalid or unsafe use.

In an embodiment an active imaging device may be attached to a control unit. The control unit will check the last sterilization date and ensure that the imaging device is no older than a predetermined safety date. If the imaging device is older than the required date, an onscreen warning will tell the user that the imaging device has expired and is unsafe for use. These features will protect the patient and the doctor from using a non-sterile imaging device.

In an embodiment a security code or some other means of identifying, and validating for use, an imaging device by a control unit maybe provided in order to verify that the imaging device is authorized for use. A validating security code or procedure of validation may be distributed to control units from a central database over the internet, by direct transfer from portable storage device such as USB device containing memory, another computer, or other storage device.

### BRIEF DESCRIPTION OF THE DRAWINGS

The features and advantages of the disclosure will become apparent from a consideration of the subsequent detailed description presented in connection with the accompanying drawings in which:

FIG. 1 is an illustration of an embodiment of the features of the disclosure and made in accordance with the teachings and principles of the disclosure;

FIG. 2 is an illustration of an embodiment of an imaging system made in accordance with the teachings and principles of the disclosure;

FIG. 3 is an illustration of an imaging system having wireless features made in accordance with the teachings and principles of the disclosure;

FIG. 4 is an illustration of an embodiment of a control unit disconnected from an imaging device, but illustrated as remaining connected to complementary apparatuses, and made in accordance with the teachings and principles of the disclosure;

FIG. 5 is an illustration of an embodiment of a control unit display made in accordance with the teachings and principles of the disclosure;

FIG. 6 is an illustration of an embodiment of a retractable display of a control unit in a retracted or closed position and made in accordance with the teachings and principles of the disclosure;

FIG. 6A is an illustration of an embodiment of a retractable display of a control unit in an open position and made in accordance with the teachings and principles of the disclosure;

FIG. 7 is a cross-sectional view of an embodiment of an imaging device head made in accordance with the teachings and principles of the disclosure;

FIG. 8 is a cross-sectional view of an embodiment of an imaging device head made in accordance with the teachings and principles of the disclosure;

FIG. 9 is a cross-sectional view of an embodiment of an imaging device head made in accordance with the teachings and principles of the disclosure;

FIG. 10 is a cross-sectional view of an embodiment of an imaging device head having a ball joint made in accordance with the teachings and principles of the disclosure;

FIG. 11 is a cross-sectional view of an embodiment of an imaging device head made in accordance with the teachings and principles of the disclosure;

FIG. 12 is a layout view of an embodiment of an imaging system made in accordance with the teachings and principles of the disclosure;

FIG. 13 is a schematic diagram of a memory of an embodiment of an imaging system made in accordance with the teachings and principles of the disclosure;

FIG. 14 illustrates an embodiment of a method in accordance with the teachings and principles of the disclosure;

FIG. 15 illustrates an embodiment of a method in accordance with the teachings and principles of the disclosure;

FIG. 16 illustrates an embodiment of a method in accordance with the teachings and principles of the disclosure;

FIG. 17 illustrates an embodiment of a method of use according to the teachings and principles of the disclosure;

FIG. 18 illustrates an embodiment of a method of reclaiming an imaging device after use according to the teachings and principles of the disclosure;

FIG. 19 illustrates an embodiment of a method of making an imaging device for use in a sterilized environment according to the teachings and principles of the disclosure;

FIG. 20 illustrates an embodiment of a method for updating an imaging device system;

FIG. 21 illustrates an embodiment of a system for providing updates to an imaging system;

FIG. 22 is a cross-sectional view of an embodiment of an imaging device head in accordance with the teachings and principles of the disclosure;

FIG. 23 is a cross-sectional view of an embodiment of an imaging device head made in accordance with the teachings and principles of the disclosure;

FIG. 24 illustrates a detailed view of an embodiment of a thermal pad with corresponding heat sink as assembled in thermal communication with surrounding heat generating components;

FIG. 25 illustrates a detailed view of an embodiment of a corresponding printed circuit board;

FIG. 26 illustrates a detailed view of an embodiment of a corresponding image sensor;

FIG. 27 illustrates a detailed view of an embodiment of a corresponding image sensor;

FIG. 28 illustrates a detailed view of an embodiment of a corresponding image sensor; and

FIG. 29 illustrates an embodiment of a method for control thermal properties and isolation of electrical components of an imaging device.

### DETAILED DESCRIPTION

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure claimed.

Before the devices, systems, methods and processes for providing and reclaiming single use imaging devices are disclosed and described, it is to be understood that this disclosure is not limited to the particular embodiments, configurations, or process steps disclosed herein as such embodiments, configurations, or process steps may vary somewhat. It is also to be understood that the terminology employed herein is used for the purpose of describing particular embodiments only and is not intended to be limiting since the scope of the disclosure will be limited only by the appended claims, if any, and equivalents thereof.

In describing and claiming the subject matter of the disclosure, the following terminology will be used in accordance with the definitions set out below.

It must be noted that, as used in this specification and the appended claims, the singular forms “a,” “an,” and “the” include plural referents unless the context clearly dictates otherwise.

As used herein, the phrase “optic mount” contemplates a structure to which optics, such as an endoscope, may be mounted or coupled thereto and a structure that may accept other optic mounting or coupling systems, such as changeable mounting systems as commonly seen in the industry.

As used herein the phrase “electrically isolate,” and any derivative thereof, contemplates electric shielding sufficient to comply with regulations in the fields of art, and must not be construed as requiring absolute isolation. For example, the phrase “electrically isolate,” when used in the medical, electrical equipment field, contemplates electric shielding sufficient to comply with International Electrotechnical Commission Standard 60601-1, including Editions 1, 2 and 3.

As used herein, the terms “comprising,” “including,” “containing,” “characterized by,” and grammatical equivalents thereof are inclusive or open-ended terms that do not exclude additional, unrecited elements or method steps.

As used herein, the phrase “consisting of” and grammatical equivalents thereof exclude any element, step, or ingredient not specified in the claim.

As used herein, the phrase “consisting essentially of” and grammatical equivalents thereof limit the scope of a claim to the specified materials or steps and those that do not materially affect the basic and novel characteristic or characteristics of the claimed disclosure.

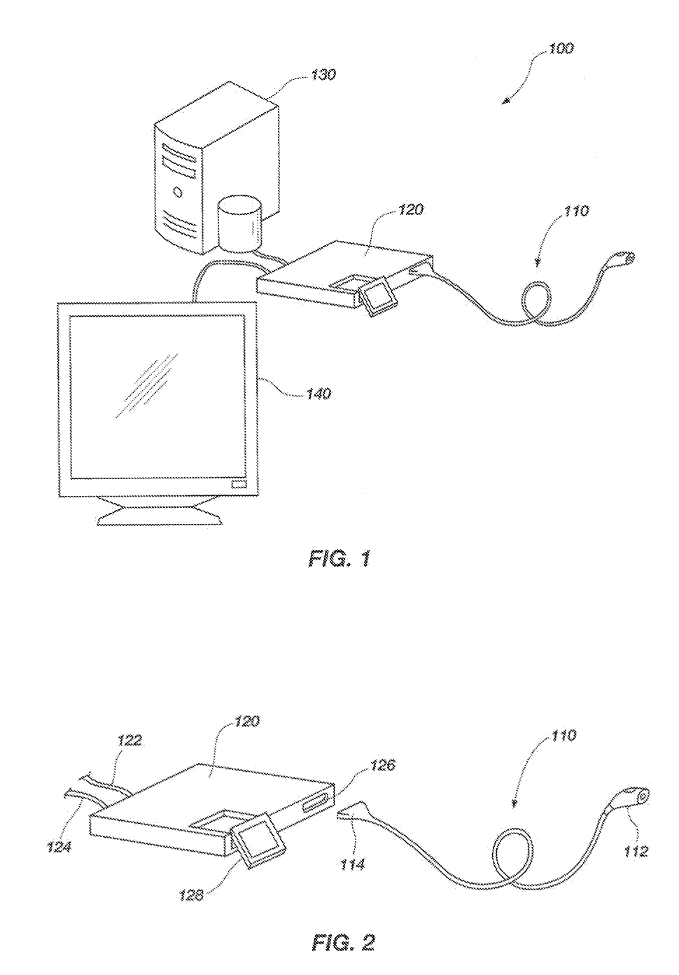

With reference primarily to FIG. 1, an embodiment of the features of the disclosure will be discussed generally. FIG. 1 illustrates a system 100 for providing a digital image using a remote imaging device 110 that may be tethered electronically and physically to a control unit 120. The control unit 120 may be configured to exchange data with imaging device 110 in order to provide single use functionality and safety in a sterile environment, such as an operating room, a doctor's office or dental office. Additionally, the control unit 120 may be electrically connected to a computer 130 or external monitor 140 for increased functionality.

Referring now to FIG. 2 where the imaging system 100 will be discussed in greater detail. As is illustrated in FIG. 2, the imaging device 110 can be connected or disconnected from the control unit 120 by way of an electronic connector 114 on the imaging device 110 that is configured to electronically and physically interact with a corresponding electronic connector 126 on the control unit 120. The ability to disconnect the imaging device 110 from the control unit 120 provides the ability to easily replace a used imaging device 110 for a sterilized, renewed imaging device 110. The imaging device 110 may have a head portion 112 generally positioned remotely from the electronic connector 114, thereby allowing greater mobility of the head portion 112 during use.

Also illustrated in FIG. 2 is an embodiment of the control unit 120 having an electronic connector 126 therein for receiving the corresponding electronic connector 114 of the imaging device 110. The control unit 120 may also have a display 128 for conveying information during a procedure to an operator or user. The display 128 may also comprise interactive functionality allowing an operator to enter commands or change what information is being displayed. Such functionality may be provided by a touch screen system as is commonly known. The control unit may also have video inputs 122 and video outputs 124 for transferring image data to other apparatuses for increased functionality. As illustrated in FIG. 1, common apparatuses may be a computer 130 or an external monitor 140.

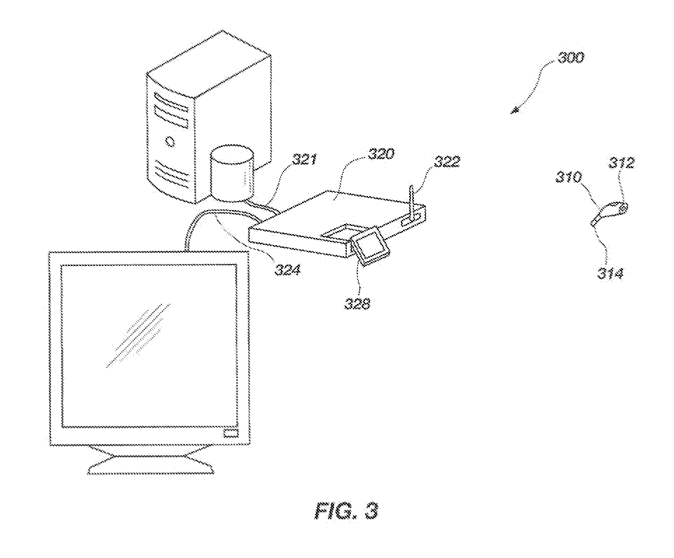

Referring now to FIG. 3 an imaging system 300 will be discussed having wireless capability and features. As is illustrated in FIG. 3, the imaging device 310 may communicate with a control unit 320 by way of wireless transmissions such as Wifi, infrared, bluetooth etc. Other forms of wireless non-tethered connectivity may also be used for providing communication between the imaging device 310 and control unit 320, including but not limited to, radio frequency from any available spectrum, infrared of all configurations, ultrasonic, and optical. The imaging device 310 may comprise a head portion 312 that houses an imaging sensor, memory and associated circuitry, which will be discussed in greater detail below. The head portion 312 may further comprise a wireless transceiver 314 for communicating with a corresponding wireless transceiver 322 housed in the control unit 320. The ability to separate the head portion 312 from the control unit 320 via wireless transmissions may provide for the easy replacement of used imaging devices for sterilized and renewed imaging devices. In other words, the wireless communication may be enabled by an electronic communication circuit that is a wireless communication transceiver configured to communicate wirelessly with a corresponding transceiver on said control unit using any of the above noted wireless technologies. The wireless functionality also allows for greater mobility of the head portion 312 during use. It will be appreciated that the wireless features and functionality may be incorporated into any of the embodiments disclosed herein or embodiments that fall within the scope of this disclosure.

Also illustrated in FIG. 3 is an embodiment of the control unit 320 having wireless capabilities and features. A transceiver 322 may be provided in or as part of the control unit 320 for receiving and transmitting wireless data to the imaging device 310. The control unit 320 may also have a display 328 for conveying information during a procedure to an operator or user. The display 328 may also comprise interactive functionality allowing an operator to enter commands or change what information is being displayed. Such functionality may be provided by a touch screen system as is commonly known. The control unit 320 may also have video inputs 321 and video outputs 324 for transferring image data to other apparatuses for increased functionality. As illustrated in FIG. 1 common apparatuses may be a computer 130 or an external monitor 140. It is within the scope of this disclosure to include an imaging system comprising both wired and wireless communication capabilities.

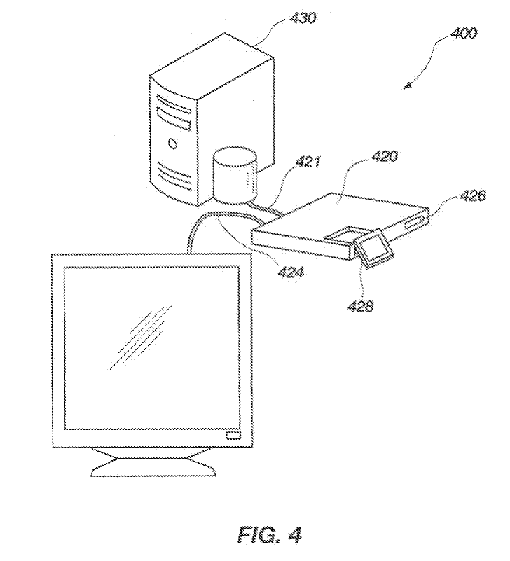

Illustrated in FIG. 4 is an embodiment of the control unit 420 disconnected from an imaging device that is illustrated as being connected to complementary apparatuses. A connector 426 may be provided therein for transferring data to and from an imaging device. The ability to separate the imaging device may provide for the easy replacement of used imaging devices with sterilized and renewed imaging devices. The control unit 420 may also have a display 428 for conveying to an operator information during a procedure. The display 428 may also comprise interactive functionality allowing an operator to enter commands or change what information is being displayed. Such functionality may be provided by a touch screen system as is commonly known. The control unit may also have video inputs 421 and video outputs 424 for transferring image data to other apparatuses for increased functionality. Common apparatuses may be a computer 430 or an external monitor 440 there by increasing the technical functionality of the system 400. A computer 430 may be used for storing the digital output from the imaging system or may be used to enhance and provide further adjustment within the system. An external monitor 440 may be used to show real time digital images to aid an operator in the use of the system, or later review and study the recorded digital imagery.

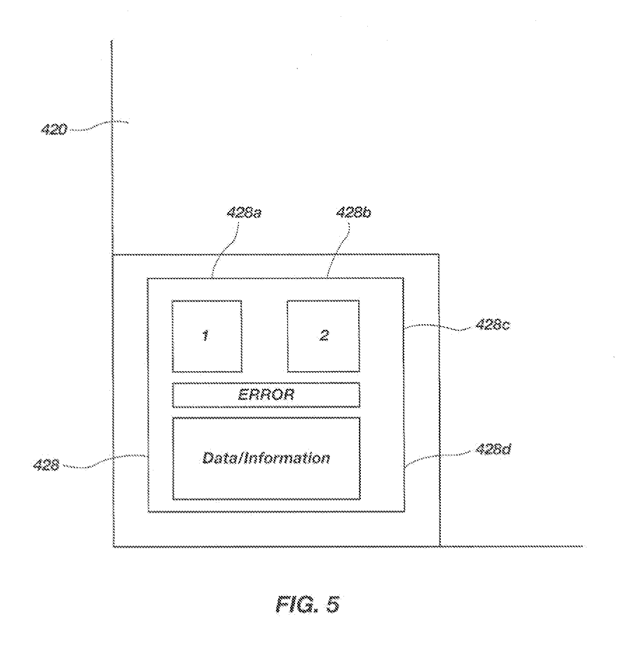

Referring now to FIG. 5 an embodiment of a control unit display 428 that may be part of a control unit 420 will be discussed in greater detail. The display 428 may be a digital display of liquid crystal design (LCD), or the display may be some other technology beside LCD, and may have touch screen functionality and capability for an operator or user to input commands into the system 400. The embodiment discussed herein may have input portions 428a and 428b whereby an operator or user may input commands into the system 400. The embodiment may further comprise a status portion 428c informing a user about the operational status of the components of the system 400. For example, display portion 428c may display an error message related to the condition of an attached imaging device 410 if the imaging device 410 has already been used or has been deemed unfit for a procedure. The display 428 may also have a dedicated message portion 428d providing instructions and further information to an operator or user. The configuration of the display 428 may change during use to accommodate further functionality. A plurality of displays 428 is contemplated by, and falls within the scope of, this disclosure and may be used alternatively or in conjunction with this embodiment. An embodiment may comprise a key pad or a button pad for control purposes within a control unit.

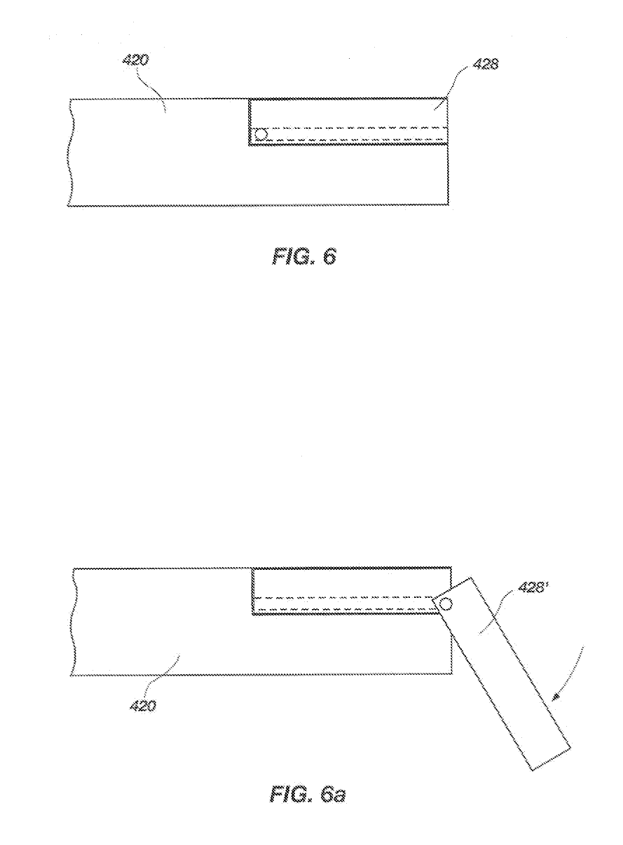

Illustrated in FIGS. 6 and 6A is an embodiment of a retractable display 428 of a control unit 420. The display 428 may have a first or retracted position within the control unit 420 (illustrated best in FIG. 6) that may be used to protect the display 428 when it is not being used. The display 428′ of FIG. 6A illustrates how the display may be deployed into a more user readable position, as it has been extended and rotated outward. As illustrated in FIGS. 6 and 6A, the display may be slid in and out of a passage and rotated about an axis to orient the display 428 in a wide range of positions.

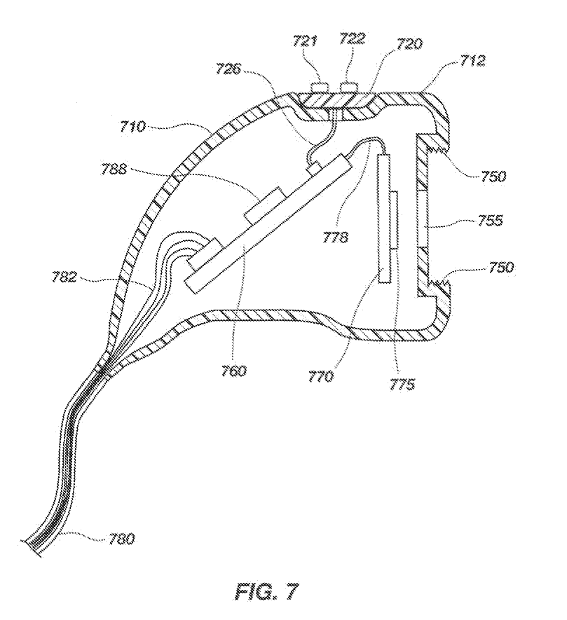

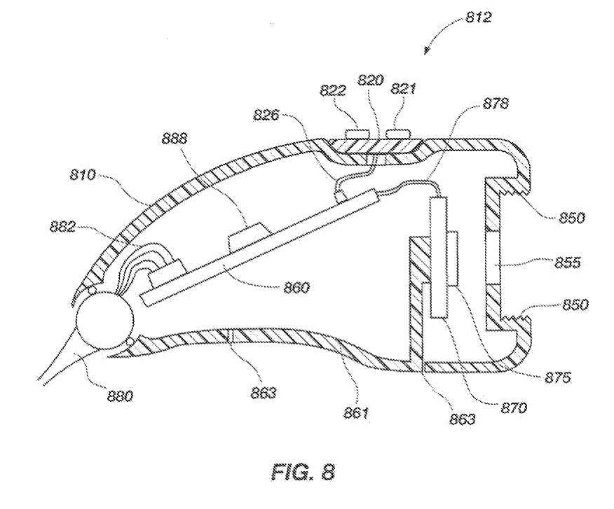

Illustrated in FIG. 7 is a cross-sectional view of an embodiment of an imaging device head 712. The imaging device head 712 may comprise a housing 710 made of a suitably rigid material, such as plastic or metal. The housing 710 may be sealed against fluids and gases so as to protect the internal circuitry and provide a suitable surface for sterilization and renewal. The imaging device head 712 may further comprise a user input panel 720 having buttons 721 and 722 for operation of the imaging device head 712. Additional, buttons may be provided and the functionality of the buttons can be customized for a given procedure or a given operator. The control panel 720 may be internally connected to other circuitry of the imaging device head 712 by an electrical connector 726.

As illustrated further in FIG. 7, imaging device head 712 may comprise an optical mount system 750, such as a C-mount system for receiving threaded accessories, for example one inch threaded accessories. A window 755 may also be incorporated into the embodiment for facilitating the transmission of light from an optical accessory to an image sensor 775. The image sensor 775 may be mounted to a supporting printed circuit board or supportive substrate 770. An electronic connector 778 may be incorporated to electronically connect the image sensor 775 to a main circuit or main printed circuit board 760. A main wiring harness 782 may be incorporated into a wired tether 780 thereby electrically connecting the components of the imaging device head 712 to a control unit.

The imaging device head 712 may further comprise a memory 788 or memory circuit allowing the storage of data within the imaging device head 712. It will be appreciated that memory may be any data storage device that is capable of recording (storing) information (data). Data that may be stored or written into memory 788 may include an identifying serial number that uniquely identifies an imaging device. Other data that maybe stored or written into memory 788 may include data such as the amount of the time the imaging device has been used, i.e., the hours of operation, or the amount of time the imaging device has been powered on. Data that may be written into memory 788 may include sterilization data or renewal data, representing the working condition of the imaging device. Data that may be stored or written into memory 788 may include data such as manufacturing date, date of last verification or quality control check, location of manufacture, i.e., may include name, city, state, street address and so forth, last control unit that the imaging device head was attached to, imaging device head diagnostic information, specific procedural settings for the imaging device head, or preferred settings for an operator or user, such as a surgeon. Data representing the above characteristics, or other indicia, of the imaging device may be recorded into memory within the imaging device.

The memory 788 may be encryption protected so as to avoid tampering or unintended use and foreseeable misuse. It should be noted that a memory 788 may be placed anywhere in the imaging device and not just the imaging device head without departing from the scope of the disclosure. The memory 788 may comprise a permanent or semi-permanent portion allowing varying degrees of data durability.

Illustrated in FIG. 8 is a cross-sectional view of an embodiment of an imaging device head 812. The imaging device head 812 may comprise a housing 810 made of a suitably rigid material such as plastic or metal. The housing 810 may be sealed against fluids and gases so as to protect the internal circuitry and provide a suitable surface for sterilization and renewal. The imaging device head 812 may further comprise a user input panel 820 having buttons 821 and 822. Additional, buttons may be provided and the functionality of the buttons can be customized for a given procedure and or a given operator. The control panel 820 maybe internally connected to other circuitry of the imaging device head 812 by an electrical connector 826.

As illustrated further in the embodiment of FIG. 8, the imaging device head 812 may comprise an optical mount system 850, such as a C-mount system for receiving threaded accessories, for example one inch threaded accessories. A window 855 may also be incorporated into the embodiment for facilitating the transmission of light from an optical accessory to an image sensor 875. The image sensor 875 may be mounted to a supporting printed circuit board or supportive substrate 870. An electronic connector 878 maybe incorporated to electronically connect the image sensor 875 to a main circuit or main printed circuit board 860. In order to provide heat dissipation from the image sensor 875 and other circuitry, a heat sink 861 may be provided. The heat sink 861 may be physically connected to the image sensor 875 and it may also be connected to the housing 810, such that heat energy can be conducted or transferred to the external portion of the imaging device head 812. The heat sink 861 may be a neutral sensor heat sink exposed externally to ensure the camera head meets cardiac floating (CF) and body floating (BF) ISO standards. An embodiment of the heat sink 861 may be made of aluminum and have fins for added heat transfer surface area. A main wiring harness 882 may be incorporated into a wired tether 880 thereby electrically connecting the components of the imaging device head 812 to a control unit.

The imaging device head 812 may further comprise a memory 888 or memory circuit allowing the storage of data within the imaging device head 812. Data that may be stored or written into memory 888 may include an identifying serial number that uniquely identifies an imaging device. Other data that may be stored or written into memory 888 may include data such as the amount of the time the imaging device has been used, i.e., the hours of operation, or the amount of time the imaging device has been powered on. Data that may be written into memory 888 may include sterilization data or renewal data, representing the working condition of the imaging device. Data that may be stored or written into memory 888 may include data such as manufacturing date, date of last verification or quality control check, location of manufacture, i.e., may include name, city, state, street address and so forth, last control unit that the imaging device head was attached to, imaging device head diagnostic information, specific procedural settings for the imaging device head, or preferred settings for an operator or user, such as a surgeon. Data representing the above characteristics, or other indicia, of the imaging device may be recorded into memory within the imaging device.

The memory 888 may be encryption protected so as to avoid tampering or unintended use and foreseeable misuse. It should be noted that a memory may be placed anywhere in the imaging device and not just the imaging device head without departing from the scope of the disclosure. The memory 888 may comprise a permanent or semi-permanent portion allowing varying degrees of data durability.

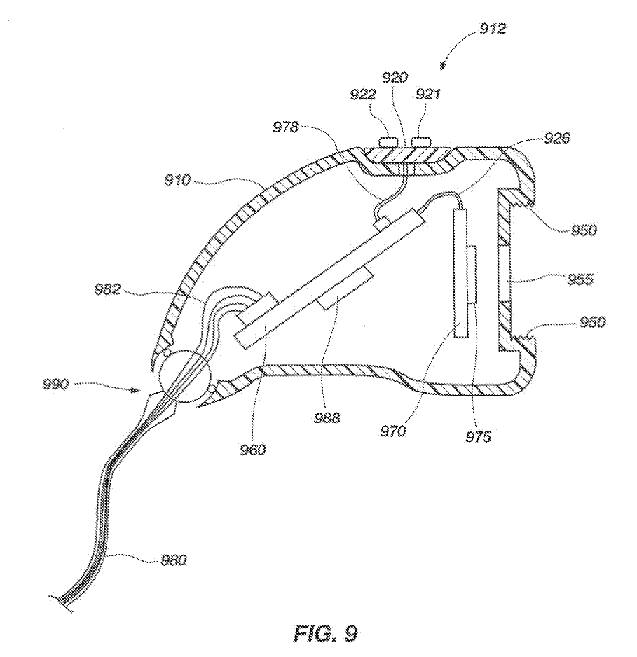

Illustrated in FIG. 9 is a cross-sectional view of an embodiment of an imaging device head 912. The imaging device head 912 may comprise a housing 910 made of a suitably rigid material such as plastic or metal. The housing 910 may be sealed against fluids and gases so as to protect the internal circuitry and provide a suitable surface for sterilization and renewal. The imaging device head 912 may further comprise a user input panel 920 having buttons 921 and 922. Additional, buttons maybe provided and the functionality of the buttons can be customized for a given procedure and or a given operator. The control panel 920 maybe internally connected to other circuitry of the imaging device head 912 by an electrical connector 926.

As illustrated further in the embodiment of FIG. 9, the imaging device head 912 may comprise an optical mount system 950, such as a C-mount system for receiving threaded accessories, for example one inch threaded accessories. A window 955 may also be incorporated into the embodiment for facilitating the transmission of light from an optical accessory to an image sensor 975. The image sensor 975 may be mounted to a supporting printed circuit board or supportive substrate 970. An electronic connector 978 may be incorporated to electronically connect the image sensor 975 to a main circuit or main printed circuit board 960. In order to provide heat dissipation from the image sensor 975 and other circuitry, a heat sink may be provided, similar to the heat sink provided in FIG. 8. The heat sink may be physically connected to the image sensor 975 and it may also be connected to the housing 910, such that heat energy can be conducted or transferred to the external portion of the imaging device head 912. A main wiring harness 982 may be incorporated into a wired tether 980 thereby electrically connecting the components of the imaging device head 912 to a control unit.

The imaging device head 912 may further comprise a memory 988 or memory circuit allowing the storage of data within the imaging device head 912. Data that may be stored or written into memory 988 may include an identifying serial number that uniquely identifies an imaging device. Other data that may be stored or written into memory 988 may include data such as the amount of the time the imaging device has been used, i.e., the hours of operation, or the amount of time the imaging device has been powered on. Data that may be stored or written into memory 988 may include data such as manufacturing date, date of last verification or quality control check, location of manufacture, i.e., may include name, city, state, street address and so forth, last control unit that the imaging device head was attached to, imaging device head diagnostic information, specific procedural settings for the imaging device head, or preferred settings for an operator or user, such as a surgeon. Data representing the above characteristics, or other indicia, of the imaging device may be recorded into memory within the imaging device.

The memory 988 may be encryption protected so as to avoid tampering or unintended use and foreseeable misuse. It should be noted that a memory may be placed anywhere in the imaging device and not just the imaging device head without departing from the scope of the disclosure. The memory 988 may comprise a permanent or semi-permanent portion allowing varying degrees of data durability.

The imaging device head 912 may comprise a ball joint 990 with a corresponding seal and socket, thereby providing increased mobility between the housing 910 and the tether 980 during articulation of the imaging device by an operator or user.

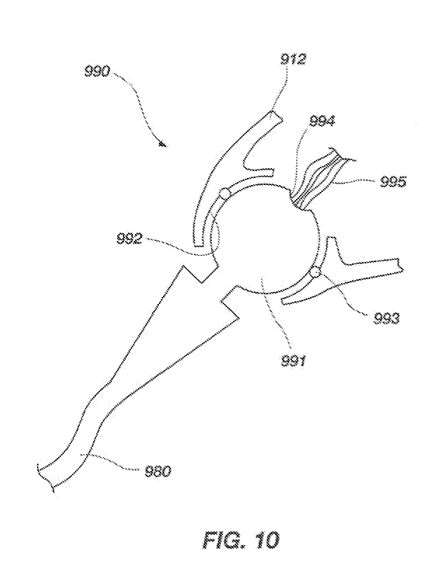

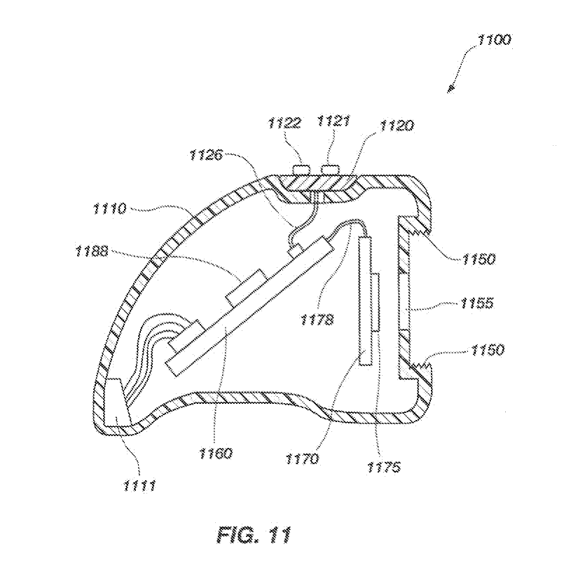

With reference primarily to FIG. 10, an embodiment of an imaging device ball joint 990 will be discussed in further detail. FIG. 10 is illustrative of a cross-sectional view of a ball joint 990, which provides greater freedom of articulation for an operator when moving the imaging device head 912 relative to the wiring tether 980. The ball joint 990 may comprise a substantially spherical rotatable portion or ball 991. The ball 991 may be configured to mechanically operate in communication with a corresponding socket 992, such that the ball 991 may substantially freely rotate while being retained within the socket 992. A seal may be provided withing the ball joint 990 by the inclusion of a seal ring 993. The seal ring 993 may also provide mechanical resistance within the ball joint 990. The ball 991 may further include an opening 994 therethrough allowing wiring 995 to pass through the ball joint 990.

With reference to FIG. 11, an embodiment of an imaging device 1100 comprising wireless transmission functionality will be discussed. A cross-sectional view of an embodiment of an imaging device head 1112 is shown in FIG. 11. The imaging device head 1112 may comprise a housing 1110 made of a suitably rigid material such as plastic or metal. The housing 1110 may be sealed against fluids and gases so as to protect the internal circuitry and provide a suitable surface for sterilization and renewal. The imaging device head 1112 may further comprise a user input panel 1120 having buttons 1121 and 1122. Additional, buttons may be provided and the functionality of the buttons can be customized for a given procedure and or a given operator. The control panel 1120 may be internally connected to other circuitry of the imaging device head 1112 by an electrical connector 1126. The imaging device head 1112 may communicate with a control unit by way of wireless transmissions such as Wifi, infrared, bluetooth etc. Other forms of wireless non-tethered connectivity may also be used for providing communication between the imaging device head 1112 and the control unit, including but not limited to, radio frequency from any available spectrum, infrared of any configuration, ultrasonic, and optical. As illustrated further in the embodiment of FIG. 11, the imaging device head 1112 may comprise an optical mount system 1150, such as a C-mount system for receiving threaded accessories, for example one inch threaded accessories. A window 1155 may also be incorporated into the embodiment for facilitating the transmission of light from an optical accessory to an image sensor 1175. The image sensor 1175 may be mounted to a supporting printed circuit board or supportive substrate 1170. An electronic connector 1178 may be incorporated to electronically connect the image sensor 1175 to a main circuit or main printed circuit board 1160. The circuitry of the imaging device head 1112 may electrically be connected to a wireless transceiver 1111 for transmitting and receiving data from a wirelessly configured control unit as illustrated in FIG. 3.

The imaging device head 1112 may further comprise a memory 1188 or memory circuit allowing the storage of data within the imaging device head 1112. Data that may be stored or written into memory 1188 may include an identifying serial number that uniquely identifies an imaging device. Other data that may be stored or written into memory 1188 may include data such as the amount of the time the imaging device has been used, i.e., the hours of operation, or the amount of time the imaging device has been powered on. Data that may be stored or written into memory 1188 may include data such as manufacturing date, date of last verification or quality control check, location of manufacture, i.e., may include name, city, state, street address and so forth, last control unit that the imaging device head was attached to, imaging device head diagnostic information, specific procedural settings for the imaging device head, or preferred settings for an operator or user, such as a surgeon. Data representing the above characteristics, or other indicia, of the imaging device may be recorded into memory within the imaging device.

The memory 1188 may be encryption protected so as to avoid tampering or unintended use and foreseeable misuse. It should be noted that a memory may be placed anywhere in the imaging device and not just the imaging device head without departing from the scope of the disclosure. The memory 1188 may comprise a permanent or semi-permanent portion allowing a varying degrees of data durability.

It will be appreciated that the ball joint illustrated in FIGS. 9 and 10 may be used by any embodiment of the disclosure without departing from the spirit or scope of the disclosure. Thus, for example, the ball joint 990 may be used with imaging device head 712, 812, 912, or 1112. Similarly, it will be appreciated that the heat sink 861 (illustrated in FIG. 8) may be used by any embodiment of the disclosure without departing from the scope of the disclosure.

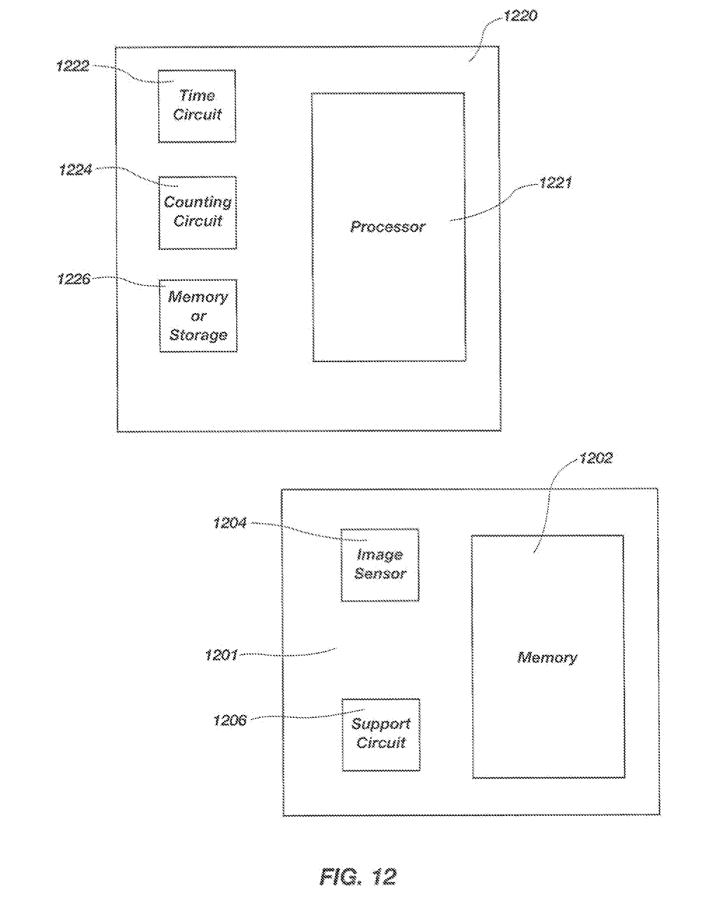

Referring now to FIG. 12 an embodiment of a system for acquiring imagery in a sterilized environment will be discussed. The system may comprise an imaging device 1201 having a memory 1202, an image sensor 1204, and supporting circuitry 1206. The system may further comprise and control unit 1220 having a processor 1221, time circuit or realtime clock 1222, a counting or incrementing circuit 1224 and a control unit memory 1226. The components will generally be provided in a housing, but are shown hear in block diagram form for simplicity and discussion purposes. It is contemplated that any of the above circuits can operate from either a control unit or an imaging device.

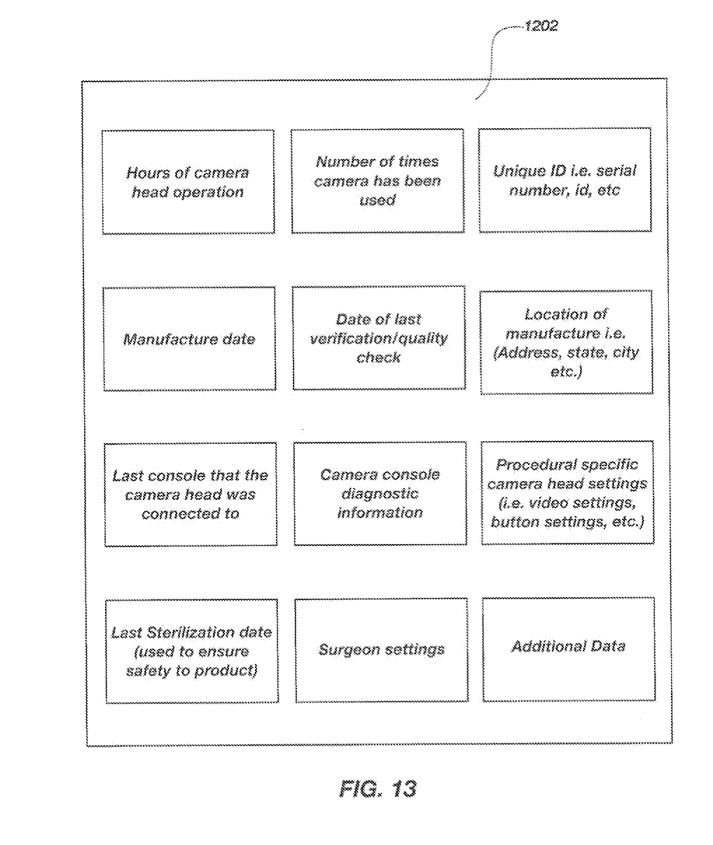

As can be seen in FIG. 13 the memory 1202 of the imaging device 1201 may comprise the following arrays of data storage:

a. Hours of camera head operation;

b. Number of times camera has been used;

c. Unique identification i.e. serial number, id, etc.;

d. Manufacture date;

e. Date of last verification/quality check;

f. Location of manufacture i.e. (Address, state, city etc.);

g. Last console that the camera head was connected to;

h. Camera console diagnostic information;

i. Procedural specific camera head settings (i.e. video settings, button settings, etc.);

j. Last Sterilization date (used to ensure safety to product); and

k. Surgeon or user settings.

Additional data may be stored within the memory 1202 that would enhance the imaging device and is considered to be within the scope of the disclosure.

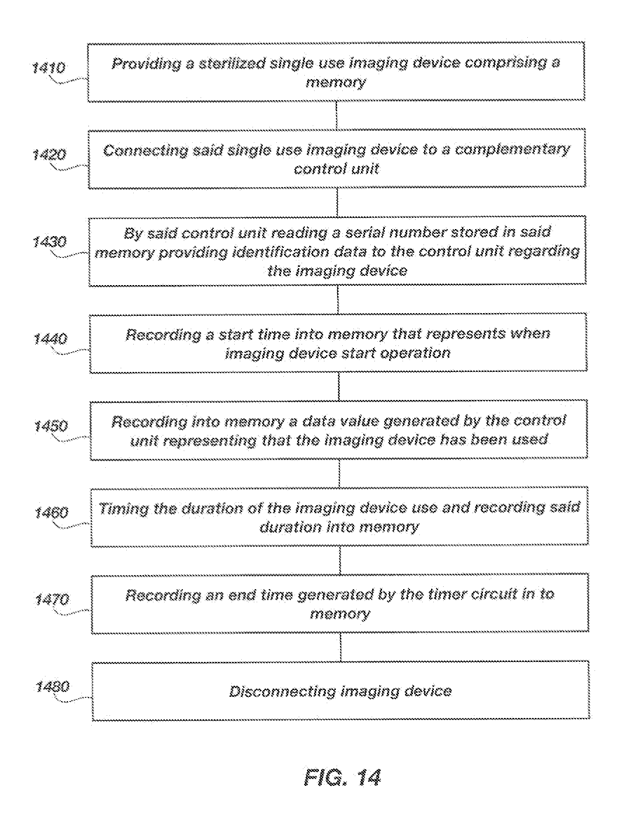

With reference to FIG. 14, a method of using an imaging system consistent with the embodiments disclosed herein will be discussed. In use, a sterilized single use imaging device 1201 will be provided that may comprise memory 1202 at 1410. At 1420 a user may connect the single use imaging device 1201 to a complementary control unit 1220 both electronically and physically. At 1430 the control unit 1220 may initiate a process of reading memory 1202 and registers the serial number of the imaging device 1201. At 1440 the system causes a value to be recorded into memory 1202 indicating that the imaging device 1201 has been used. At 1450 the system records into memory 1202 the date and time the imaging device 1201 is connected to the control unit 1220. At 1460 a timing process is initiated by the control unit from the base line time recorded at 1450 and tracks or times the duration that the imaging device 1201 is used and the duration is recorded into memory 1202 at 1470. After use, the imaging device 1201 is disconnected from the control unit 1220 at 1480 and then discarded for renewal or reclamation.

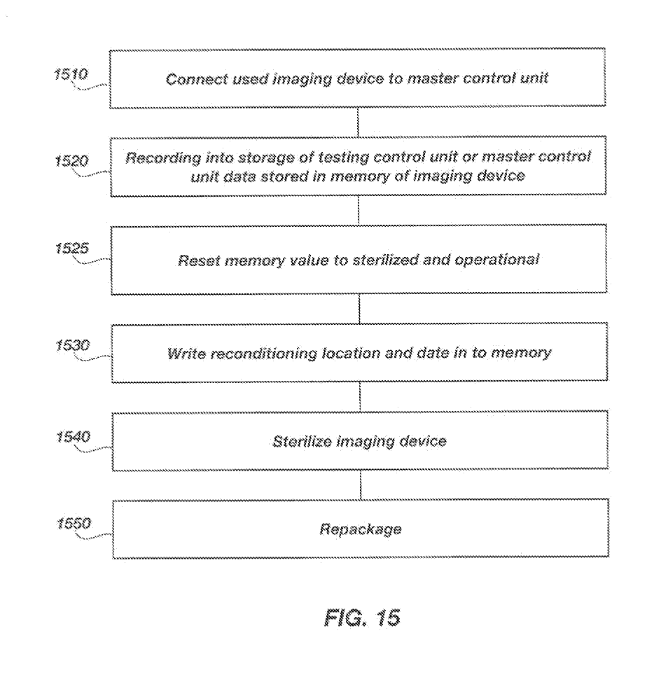

Referring now to FIG. 15, a method of renewing and reclaiming a single use imaging device 1201 will be discussed. At 1510 the imaging device 1201 may be connected to a testing control unit or a master control unit. At 1520 the testing control unit or master control unit causes the data stored in memory 1202 to be recorded into storage on the testing control unit or master control unit as stored, in order for the specific imaging device 1201 to be renewed. At 1525 a value is placed in memory 1202 indicating that the imaging device has been renewed and is ready for use such that when connected to another control unit for use it will operate. The location and date of the renewal may then be recorded into memory 1202 at 1530. At 1540 the imaging device 1201 can be sterilized and (at 1550) placed in a protective sterilized package.

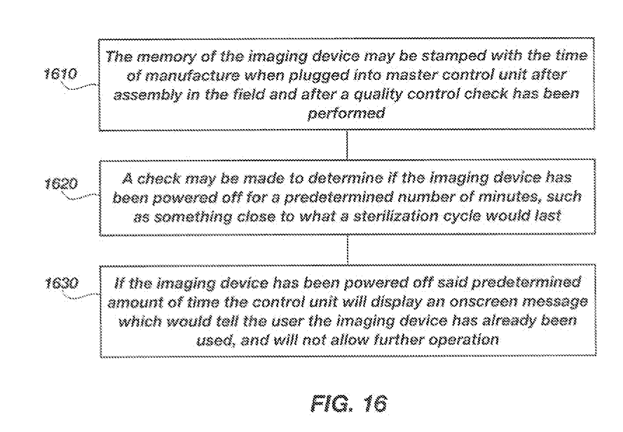

With reference to FIG. 16 an alternative embodiment of a method of use will be discussed illustrating safety settings of the embodiment. At 1610 the memory imaging device head may be stamped with time of manufacture when it is plugged into the master control unit or master console after assembly in the field, i.e., in an operating room, and after a quality control check has been performed. At 1620 a check may be made to determine if the imaging device has been powered off for a predetermined number of minutes, such as a time frame that is close to what a typical sterilization cycle would last. At 1630, if the imaging device has been powered off the predetermined amount of time the control unit will display an onscreen message telling the user the imaging device has already been used, and will not allow further operation, such that no image will be produced through video feed. This feature will ensure the imaging device, i.e., the camera, will not be used more than one time per sterilization cycle. This feature also protects the patient and the doctor from an invalid or unsafe use and foreseeable misuse.

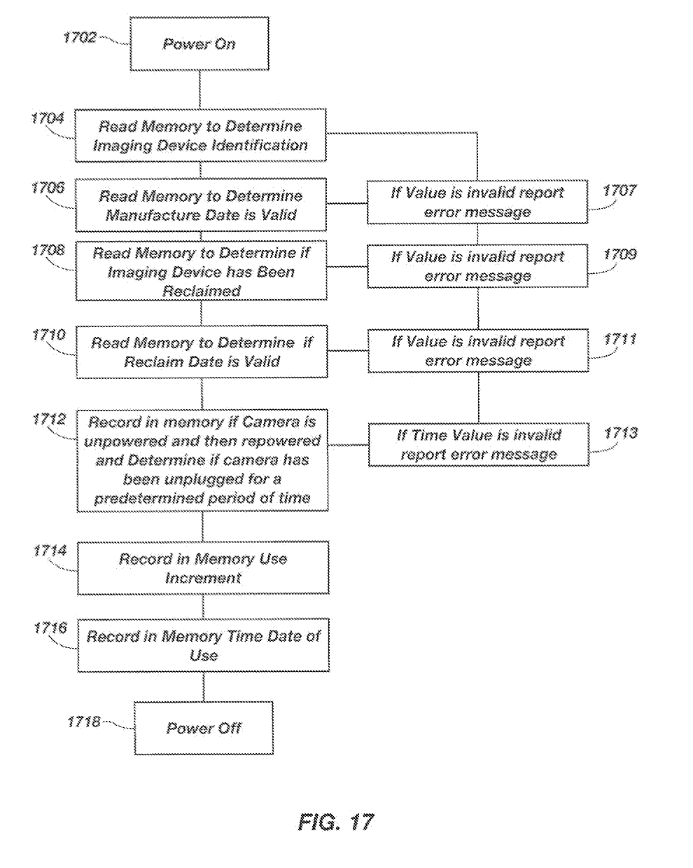

Referring to FIG. 17 an embodiment of a method of use will be discussed. During use, an imaging device may be connected to a control unit. Upon connection, an electronic communication connection is formed between the imaging device and the control unit. At 1702 the imaging device may be powered on by power supplied by the control unit. At 1704 a processor in the control unit may cause data regarding imaging device identification that may be stored in a memory within the imaging device to be read. At 1706 a processor in the control unit may cause data regarding the manufacturing date of the imaging device to be read from memory within the imaging device. The processor in the control unit may then compare the data to a predetermined data value range. At 1707 an error message may be displayed if the read data is outside the predetermined data value range and the imaging device will be stopped from operating. At 1708 a processor in the control unit may cause data regarding the reclamation of the imaging device to be read from memory within the imaging device. The data regarding reclamation of the imaging device may include data representing whether or not the imaging device has been previously used. The processor may then compare the data to a predetermined data value range. At 1709 an error massage may be displayed if the read data is outside the predetermined data value range and the imaging device will be stopped from operating. At 1710 a processor in the control unit may cause data regarding the reclamation date of the imaging device to be read from memory within the imaging device. The processor may then compare the data to a predetermined data value range. At 1711 an error massage may be displayed if the read data is outside the predetermined data value range and the imaging device will be stopped from operating. At 1712 a processor in the control unit may cause usage information of the current procedure to be monitored to note whether imaging device has been unpowered for a predetermined period of time and then re-powered. If this condition occurs it is possible that the imaging device has been tampered with or that an attempt has been made to sterilize the imaging device and use it a second time. The predetermined period of time may correspond to the amount of time a typical sterilization process would normally take. The processor then compares the data to a predetermined data value range. At 17013 an error massage may be displayed if the data read is outside the predetermined data value range and the imaging device will be stopped from operating. At 1714 a processor in the control unit may cause a value to be placed in memory in the imaging device indicating that the imaging device has been used. At 1716 a processor in the control unit may cause the date and time of use to be recorded in memory in the imaging device. Additional information may be recorded into the memory of the imaging device such as, for example, duration of use, procedure settings, and user settings and any other data suitable for recording to memory. The imaging device may be disconnected from the control unit and thereby powered off at 1718.

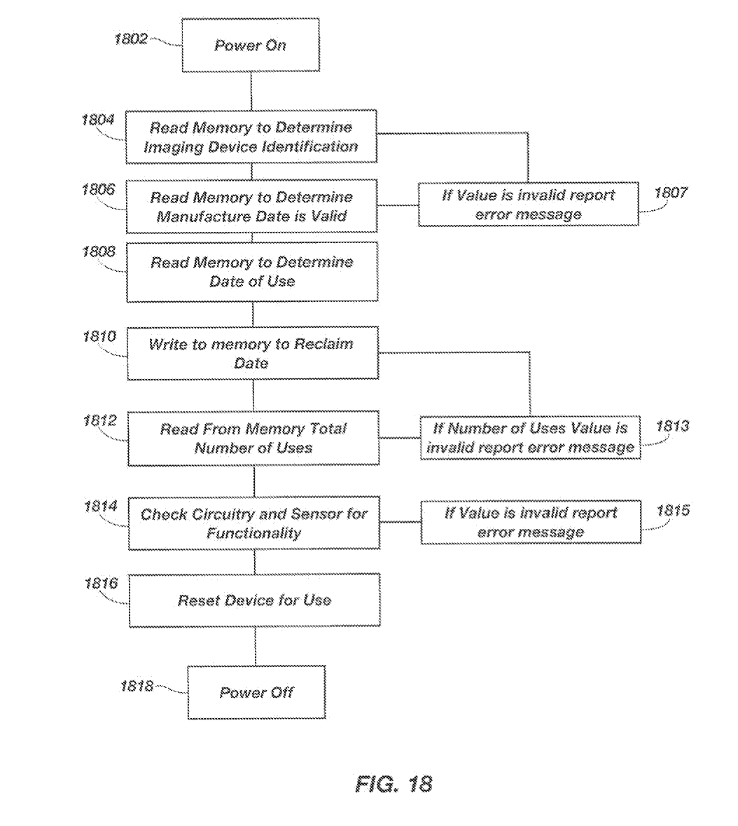

Referring now to FIG. 18 a method of reclaiming an image device after use will be discussed. It should be noted that a single use imaging device may comprise the durability to be used a plurality of times, however sterilization requirements may prevent an imaging device from being used more than once without a process for reclaiming the imaging device, thereby returning it to a sterilized condition. A method of reclamation for an imaging device may comprise the process of powering on the imaging device at 1802, when the imaging device is electrically connected to a control unit. At 1804 a processor in the control unit may cause data representing identification information for the imaging device to be stored in storage in the control unit. A control unit maybe a master control unit configured for reclaiming the imaging devices. The master control unit may track a plurality of imaging devices thereby keeping a catalog of associated information such as use and condition of the device or devices. At 1806 a processor in the control unit may cause that data representing a manufacturing date to be read and compared to a predetermined value or range of values. If the read data is out of the predetermined range value, an error report may be issued at 1807. At 1808 a processor in the control unit may cause data representing use data written in memory of the imaging device to be read and recorded into storage in the control unit. At 1810 a processor may cause data representing a date and time of reclamation to be recorded into memory in the imaging device. At 1812 a processor in the control unit may cause that data representing the number of uses of the imaging device to be read and recorded into storage in the control unit. The processor may compare the read data to a predetermined value or range of values to determine whether the imaging device is fit for continued use. If the predetermined value is exceeded an error message may be displayed (at 1813) and the imaging device may be retired. At 1814 a processor in the control unit may initiate a test or quality control check of all the circuitry in the imaging device to ensure that the device is functional. At 1815 it may be determined that the imaging device failed the quality control check and an error massage may be displayed. At 1816 the imaging device can be reset for use. The resetting process may comprise writing data to the memory of the imaging device indicating that the imaging device has been reclaimed and sterilized. At 1816 the device may be disconnected from the control unit and physically sterilized and repackaged.

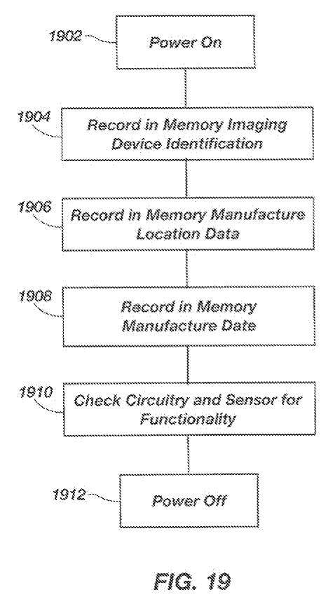

With reference primarily to FIG. 19 an embodiment of a method for making an imaging device having memory therein for use in a sterilized environment will be discussed. At 1902 an imaging device may be powered on upon being connected to a control unit. The control unit may be a master control unit configured for the manufacturing process. At 1904 a processor in the control unit may cause that data representing an identification serial number for the imaging device to be written into memory of the imaging device. At 1906 a processor in the control unit may cause that data representing the location of manufacture be recorded to memory in the imaging device. At 1908 a processor may cause that data representing the date of manufacture may be recorded into memory on the imaging device. At 1910 a processor in the control unit may initiate a test or quality control check of all the circuitry in the imaging device to ensure that the device is functional. At 1912 the imaging device may be unplugged from the control and sterilized for packaging.

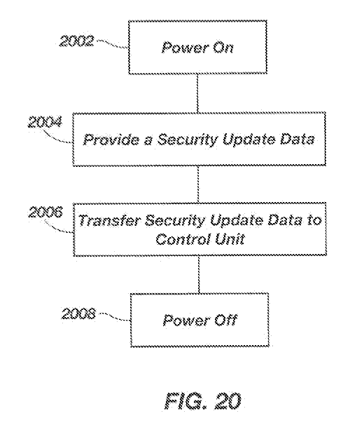

Referring to an embodiment illustrated in FIG. 20, a system having a security code or some other means of identifying, and validating for use, an imaging device by a control unit, in order to verify that the imaging device is authorized for use will now be described. A validating security code or procedure of validation may be distributed to control units from a central database over the internet, by direct transfer from portable storage device such as USB device containing memory, another computer, or other storage device. With reference to FIG. 20 an embodiment of a method for providing updates with in a medical imaging system will be discussed. At 2002 a control unit may be powered on to receive a security update. At 2004 security update data may provided comprising validation codes that correspond to imaging devices to be connected to the control unit. Such validation codes may enable the system to insure that users of the system may be prevented from using imaging devices that have been selected for non-use by a manufacturer or distributor. Selection criteria for non-use may include safety considerations, recall considerations, anti counterfeit measures, and sales and contract considerations. At 2006 the data may be transferred into storage or memory of the control unit in order to provide that data for later comparison to security codes provided by imaging devices. It is within the scope of this disclosure to include all means for transferring data, including but not limited to, transmission over a network, transfer via on site transmission from a storage medium that is portable, such as a disk, memory drive, or short distance wireless transmission. At 2008 the system may be powered off.

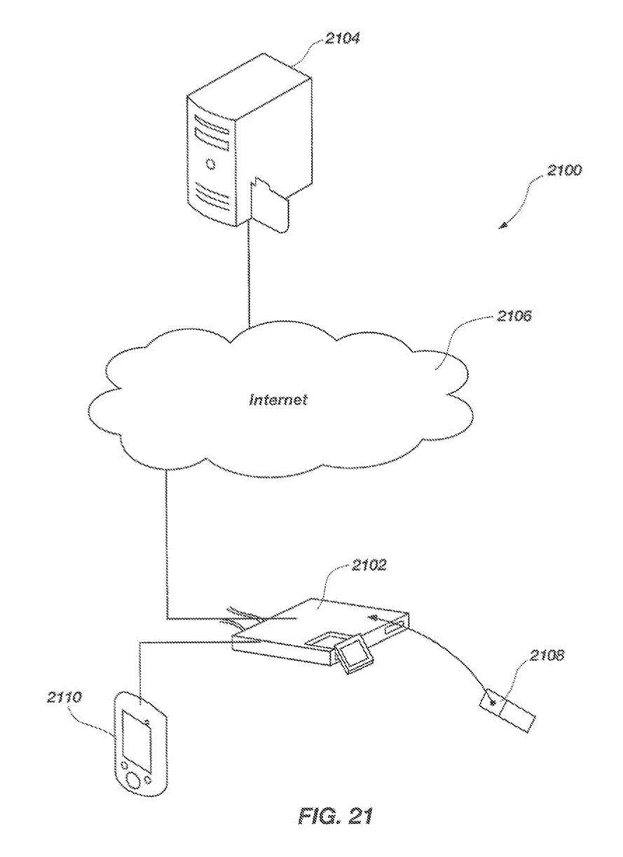

With reference primarily to FIG. 21 an embodiment of an imaging system have the feature of updating data will be discussed. An imaging system 2100 may comprise a control unit 2102 and a data server 2104. The control unit 2106 may be electronically in communication with the data server 2104 over a network such as the internet 2106. The control unit 1202 may receive update data over the internet 2106 from data server 2104. The control unit 2102 may also receive update data directly from a memory transfer device 2108 such as a memory stick, thumb drive, jump drive, hard drive, optical disk to name a few. The control unit 2102 may also receive update data from another computer or portable device 2110 such as a PDA or laptop that is presented to the control unit 2102 on site. Data transfer may be made with a physical connection and or by a wireless transfer of data.

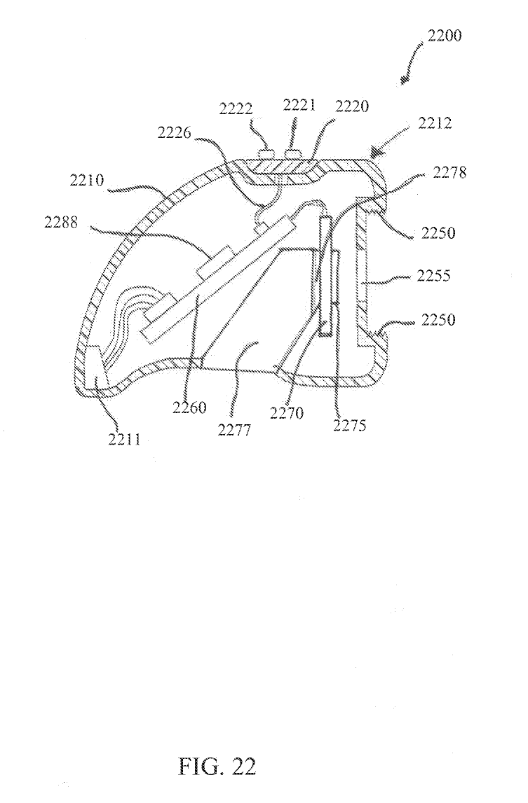

With reference to FIG. 22, an embodiment of an imaging device 2200 comprising a heat sink 2277 having a thermal pad 2278 will be discussed. A cross-sectional view of an embodiment of an imaging device head 2212 is shown in FIG. 22. The imaging device head 2212 may comprise a housing 2210 made of a suitably rigid material such as a plastic or metal. The housing 2210 may be sealed against fluids and gases so as to protect the internal circuitry and provide a suitable surface for sterilization and renewal. The imaging device head 2212 may further comprise a user input panel 2220 having buttons 2221 and 2222. Additional, buttons may be provided and the functionality of the buttons can be customized for a given procedure and/or a given operator. The control panel 2220 may be internally connected to other circuitry of the imaging device head 2212 by an electrical connector 2226. The imaging device head 2212 may communicate with a control unit by way of wireless transmissions such as Wifi, infrared, bluetooth etc. Other forms of wired and wireless non-tethered connectivity may also be used for providing communication between the imaging device head 2212 and the control unit, including but not limited to, hard wired, radio frequency from any available spectrum, infrared of any configuration, ultrasonic, and optical.

As illustrated further in the embodiment of FIG. 22, the imaging device head 2212 may comprise an optical mount system 2250, such as a C-mount system for receiving threaded accessories, for example one inch threaded accessories. A window 2255 may also be incorporated into the embodiment for facilitating the transmission of light from an optical accessory to an image sensor 2275. The image sensor 2275 may be mounted to a supporting printed circuit board or supportive substrate 2270. An electronic connector 2278 may be incorporated to electronically connect the image sensor 2275 to a main circuit or main printed circuit board 2260. The circuitry of the imaging device head 2212 may electrically be connected to a wireless transceiver 2211 for transmitting and receiving data from a wirelessly configured control unit as illustrated in FIG. 3.

The imaging device head 2212 may further comprise a memory 2288 or memory circuit allowing the storage of data within the imaging device head 2212. Data that may be stored or written into memory 2288 may include an identifying serial number that uniquely identifies an imaging device. Other data that may be stored or written into memory 2288 may include data such as the amount of the time the imaging device has been used, i.e., the hours of operation, or the amount of time the imaging device has been powered on. Data that may be stored or written into memory 2288 may include data such as manufacturing date, date of last verification or quality control check, location of manufacture, i.e., may include name, city, state, street address and so forth, last control unit that the imaging device head was attached to, imaging device head diagnostic information, specific procedural settings for the imaging device head, or preferred settings for an operator or user, such as a surgeon. Data representing the above characteristics, or other indicia, of the imaging device may be recorded into memory within the imaging device.

The memory 2288 may be encryption protected so as to avoid tampering or unintended use and foreseeable misuse. It should be noted that a memory may be placed anywhere in the imaging device and not just the imaging device head without departing from the scope of the disclosure. The memory 2288 may comprise a permanent or semi-permanent portion allowing varying degrees of data durability.

In order to provide protection to a user against electrical contact an embodiment may be electrically sealed or electrically insulated from a user. It will be appreciated that a user may be a surgeon, a surgical assistant, a technician, a patient, or any other person who may come in contact with the device. Such insulation may provide for heat transfer while at the same time electrically insulating the user from the electronics of the camera head 2200. Illustrated in FIG. 22 is a heat sink 2277 having a thermal pad 2278. The thermal pad 2278 maybe configured to allow the transfer of thermal energy from circuit board 2270 and image sensor 2275 to the heat sink 2277, while electrically insulating the components from the heat sink 2277, thereby dissipating heat generated by the circuitry without risk of electrical contact with the user. The thermal pad 2278 may be substantially rigid or may be deformable. The thermal pad 2278 may be configured to cover substantially all of the surface contact area between the heat sink 2277 and any heat generating circuitry. The thermal pad may be configured to cover a portion of the surface contact area between the heat sink 2277 and any heat generating circuitry. The thermal pad 2278 may be configured to cover a plurality of surface contact areas. An embodiment may comprise a plurality of thermal pads 2278 working on a single surface contact area. An embodiment may comprise a plurality of thermal pads 2278 working on a plurality of surface contact areas. An embodiment may comprise a thermal pad 2278 having areas of varying thickness configured to accommodate the structure and geometry of surrounding components. An embodiment may comprise a thermal pad 2278 comprising a plurality of materials. An embodiment may comprise a thermal pad 2278 comprising fold lines.

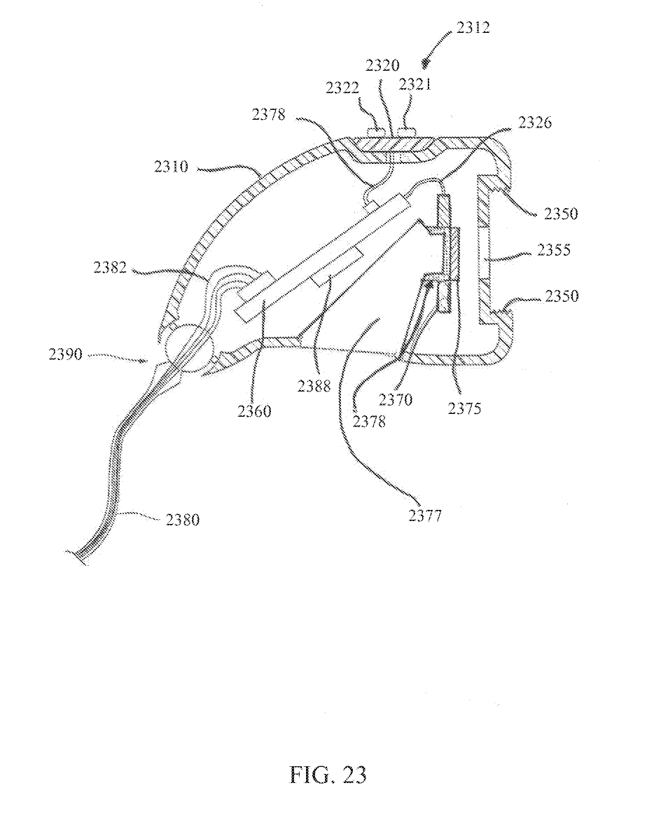

As illustrated further in the embodiment of FIG. 23, the imaging device head 2312 may comprise an optical mount system 2350, such as a C-mount system for receiving threaded accessories, for example one inch threaded accessories. A window 2355 may also be incorporated into the embodiment for facilitating the transmission of light from an optical accessory to an image sensor 2375. The image sensor 2375 may be mounted to a supporting printed circuit board or supportive substrate 2370. An electronic connector 2378 may be incorporated to electronically connect the image sensor 2375 to a main circuit or main printed circuit board 2360.

The imaging device head 2312 may further comprise a memory 2388 or memory circuit allowing the storage of data within the imaging device head 2312. Data that may be stored or written into memory 2388 may include an identifying serial number that uniquely identifies an imaging device. Other data that may be stored or written into memory 2388 may include data such as the amount of the time the imaging device has been used, i.e., the hours of operation, or the amount of time the imaging device has been powered on. Data that may be stored or written into memory 2388 may include data such as manufacturing date, date of last verification or quality control check, location of manufacture, i.e., may include name, city, state, street address and so forth, last control unit that the imaging device head was attached to, imaging device head diagnostic information, specific procedural settings for the imaging device head, or preferred settings for an operator or user, such as a surgeon. Data representing the above characteristics, or other indicia, of the imaging device may be recorded into memory within the imaging device.

The memory 2388 may be encryption protected so as to avoid tampering or unintended use and foreseeable misuse. It should be noted that a memory may be placed anywhere in the imaging device and not just the imaging device head without departing from the scope of the disclosure. The memory 2388 may comprise a permanent or semi-permanent portion allowing a varying degrees of data durability.

In order to provide protection to a user against electrical contact an embodiment may be electrically sealed or electrically insulated from a user. Such insulation may provide for heat transfer while at the same time electrically insulating the user from the electronics of the camera head 2300 and camera control unit (CCU). Illustrated in FIG. 23 is a heat sink 2377 having a thermal pad 2378. The thermal pad 2378 may be configured to allow the transfer of thermal energy from circuit board 2370 and image sensor 2375 to the heat sink 2377 while electrically insulating the components from the heat sink 2377, thereby dissipating heat generated by the circuitry without risk of electrical contact with the user. The thermal pad 2378 may be substantially rigid or may be deformable. The thermal pad 2378 may be configured to cover substantially all of the surface contact area between the heat sink 2377 and any heat generating circuitry. The thermal pad may be configured to cover a portion of the surface contact area between the heat sink 2377 and any heat generating circuitry. The thermal pad 2378 may be configured to cover a plurality of surface contact areas. An embodiment may comprise a plurality of thermal pads 2378 working on a single surface contact area. An embodiment may comprise a plurality of thermal pads 2378 working on a plurality of surface contact areas. An embodiment may comprise a thermal pad 2378 having areas of varying thickness configured to accommodate the structure and geometry of surrounding components. An embodiment may comprise a thermal pad 2378 comprising a plurality of materials. An embodiment may comprise a thermal pad 2378 comprising fold lines.

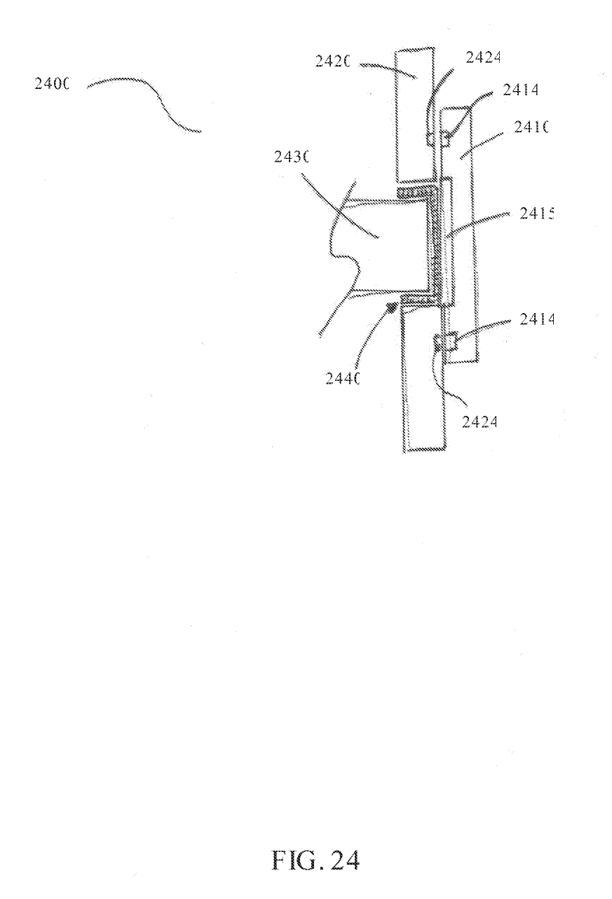

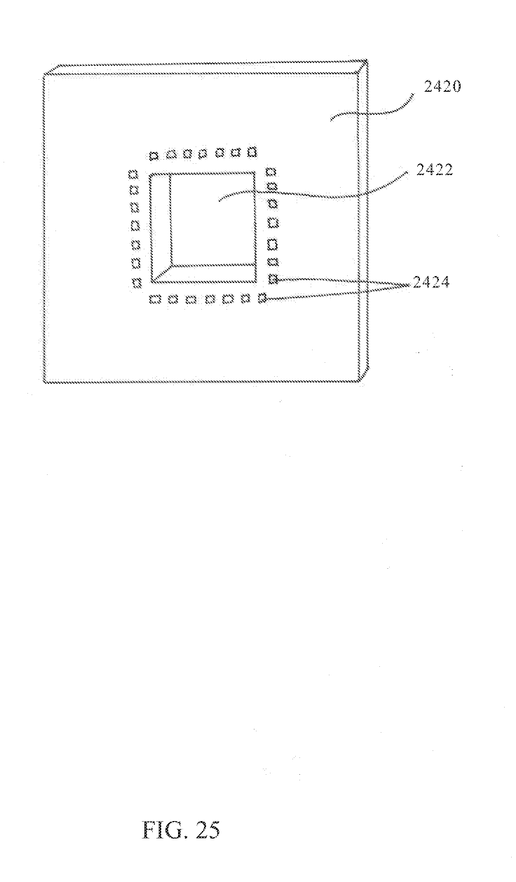

Illustrated in FIG. 24 is a detailed view of an embodiment of a thermal pad 2440 with corresponding heat sink 2430 as assembled in thermal communication with surrounding heat generating components. The assembly 2400 may comprise an imaging sensor or chip 2410, a printed circuit board 2420, a heat sink 2430 and a thermal pad 2440. Referring to FIGS. 24 and 25, the printed circuit board 2420 may comprise an opening 2422 for receiving the heat sink 2430 and may comprise a plurality of electrical contacts 2424 for contacting corresponding sensor contacts 2414 on the imaging sensor 2410.

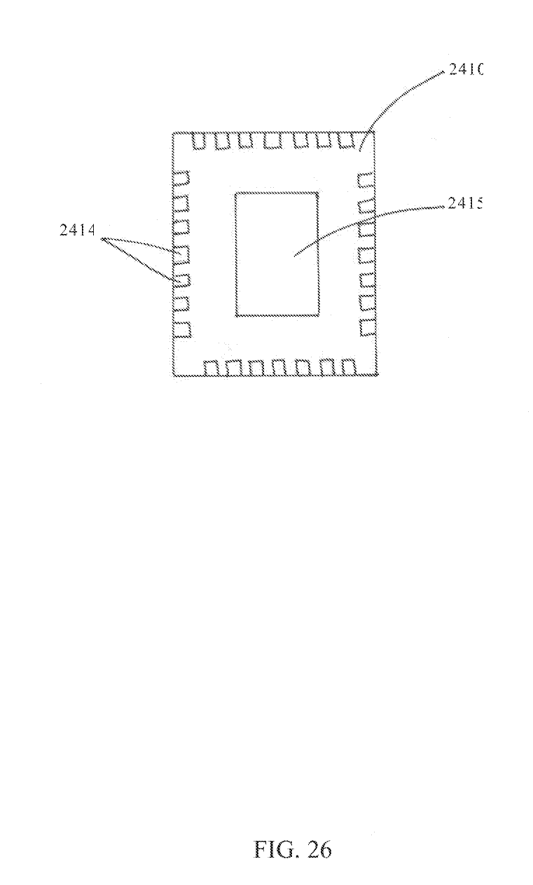

Referring to FIGS. 24 and 26, the imaging sensor 2410 comprises a thermal pad 2415 used to dissipate and direct heat from the imaging sensor 2410. The thermal interface material 2440 illustrated in FIG. 24 is located between the heat sink 2430 and the thermal pad 2415. The thermal interface 2440 is a material that is thermally conductive, but electrically non-conductive or insulating. The insulating properties of the material 2440 prevent electrical current from flowing from the electronics in the imaging device or camera head to the heat sink 2430, which may be in contact with the patient or user of the device or both. It will be appreciated that thermal energy may be dissipated from the imaging sensor 2410 through the thermal pad 2415 and through the thermal pad 2440 to the heat sink 2430 where the heat is quickly dissipated without the flow of electricity. The heat sink 2430 may be any heat sink known in the art and may be made of a thermally conductive material to quickly distribute and dissipate the heat from the sensor 2410. The thermal pad 2440 may be substantially rigid or may be deformable. The thermal pad 2440 may be configured to cover substantially all of the surface contact area between the heat sink 2430 and any heat generating circuitry. The thermal pad may be configured to cover a portion of the surface contact area between the heat sink 2430 and any heat generating circuitry. The thermal pad 2440 may be configured to cover a plurality of surface contact areas. An embodiment may comprise a plurality of thermal pads 2440 working on a single surface contact area. An embodiment may comprise a plurality of thermal pads 2440 working on a plurality of surface contact areas. An embodiment may comprise a thermal pad 2440 having areas of varying thickness configured to accommodate the structure and geometry of surrounding components. An embodiment may comprise a thermal pad 2440 comprising a plurality of materials. An embodiment may comprise a thermal pad 2440 comprising fold lines.

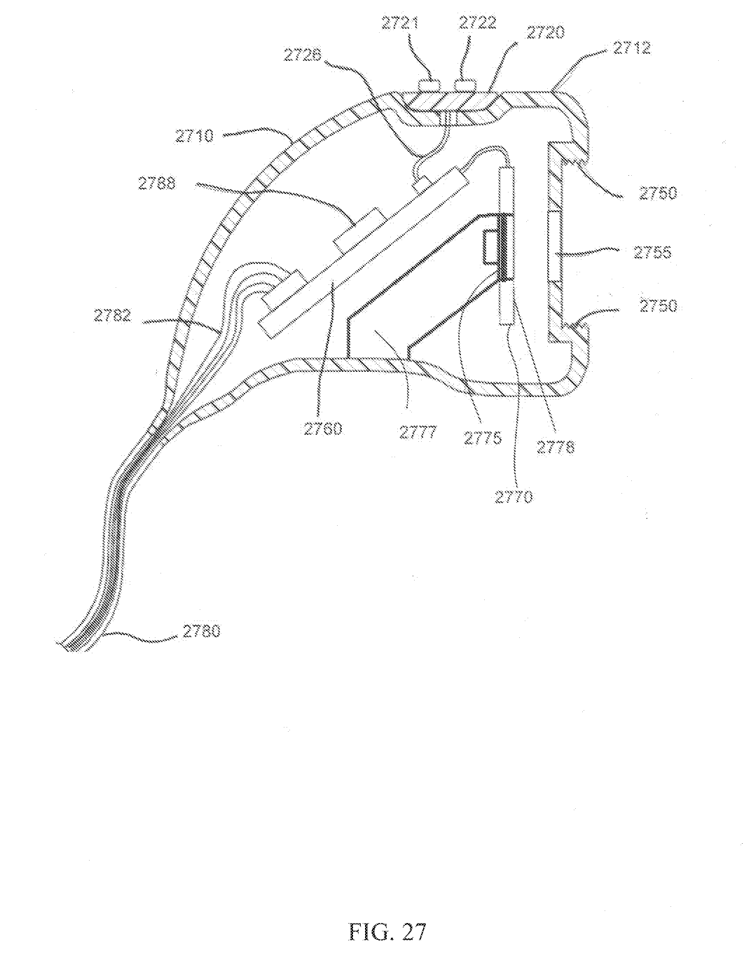

Illustrated in FIG. 27 is a cross-sectional view of an embodiment of an imaging device head 2712. The imaging device head 2712 may comprise a housing 2710 made of a suitably rigid material, such as plastic or metal. The housing 2710 may be sealed against fluids and gases so as to protect the internal circuitry and provide a suitable surface for sterilization and renewal. The imaging device head 2712 may further comprise a user input panel 2720 having buttons 2721 and 2722 or electrical communication mechanisms for operation of the imaging device head 2712. Additional buttons or electrical communication mechanisms may be provided and the functionality of the buttons or electrical communication mechanisms can be customized for a given procedure or a given operator. The control panel 2720 may be internally connected to other circuitry of the imaging device head 2712 by an electrical connector 2726.

As illustrated further in FIG. 27, imaging device head 2712 may comprise an optical mount system 2750, such as a C-mount system for receiving threaded accessories, for example one inch threaded accessories. A window 2755 may also be incorporated into the embodiment for facilitating the transmission of light from an optical accessory to an image sensor 2775. The image sensor 2775 may be mounted to a supporting printed circuit board or supportive substrate 2770. An electronic connector 2778 may be incorporated to electronically connect the image sensor 2775 to a main circuit or main printed circuit board 2760. A main wiring harness 2782 may be incorporated into a wired tether 2780 thereby electrically connecting the components of the imaging device head 2712 to a control unit.

The imaging device head 2712 may further comprise a memory 2788 or memory circuit allowing the storage of data within the imaging device head 2712. It will be appreciated that memory may be any data storage device that is capable of recording (storing) information (data). Data that may be stored or written into memory 2788 may include an identifying serial number that uniquely identifies an imaging device. Other data that may be stored or written into memory 2788 may include data such as the amount of the time the imaging device has been used, i.e., the hours of operation, or the amount of time the imaging device has been powered on. Data that may be written into memory 2788 may include sterilization data or renewal data, representing the working condition of the imaging device. Data that may be stored or written into memory 2788 may include data such as manufacturing date, date of last verification or quality control check, location of manufacture, i.e., may include name, city, state, street address and so forth, last control unit that the imaging device head was attached to, imaging device head diagnostic information, specific procedural settings for the imaging device head, or preferred settings for an operator or user, such as a surgeon. Data representing the above characteristics, or other indicia, of the imaging device may be recorded into memory within the imaging device.

The memory 2788 may be encryption protected so as to avoid tampering or unintended use and foreseeable misuse. It should be noted that a memory 2788 may be placed anywhere in the imaging device and not just the imaging device head without departing from the scope of the disclosure. The memory 2788 may comprise a permanent or semi-permanent portion allowing varying degrees of data durability. An embodiment may further comprise a heat sink 2777 for transferring heat away from circuitry and said image sensor 2775, and a thermal pad 2778 configured to be in physical contact with said image sensor 2775 and said circuitry and said heat sink 2777. The thermal pad 2778 may be configured to electronically isolate the image sensor 2775 from the patient and operator while enhancing the dissipation of heat generated by the image sensor 2775. As can be seen in the figure, the thermal pad 2778 may be disposed between the image sensor 2775, or the non-light sensing portion of the image sensor 2775, and the heat sink 2777. As illustrated, the thermal pad 2778 may contact the image sensor 2775 or the heat sink 2777 at a plurality of points. It is within the scope of this disclosure to contemplate a multi piece thermal pad that may be place under the plurality of contact areas between the heat sink 2777 and the image sensor 2775. The thermal pad 2778 may also contact the image sensor 2775 at less than the entirety of the surface area of the image sensor. The thermal pad 2778 may be larger than the area of either the heat sink 2777 or the area of the image sensor 2775.

An embodiment of a single use imaging device for use with and communicating with a control unit may comprise a housing, an image sensor, an opening proximate to an optic mount that has been configured to facilitate the transmission of light from any attached optics to said image sensor. The imaging device may further comprise a memory for storing data representing any characteristics of the imaging device. In order to manage the heat created by the electronics in the imaging device, the embodiment may further comprise a heat sink for transferring heat away from circuitry and said image sensor, and a thermal pad configured to be in physical contact with said image sensor and said circuitry and said heat sink. Wherein said thermal pad is further configured to electrically isolate said image sensor and said circuitry from said heat sink, and is further configured to thermally conduct heat generated by said circuitry and said image sensor. The thermal pad may be disposed between said image sensor and said heat sink, such that the image sensor is electrically isolated from said heat sink. The thermal pad may also be in substantial contact with said image sensor across substantially all of the surface area defined by the image sensor. In other configurations the thermal pad may be in substantial contact with said image sensor at a portion that is less than the surface area defined by the imaging sensor.

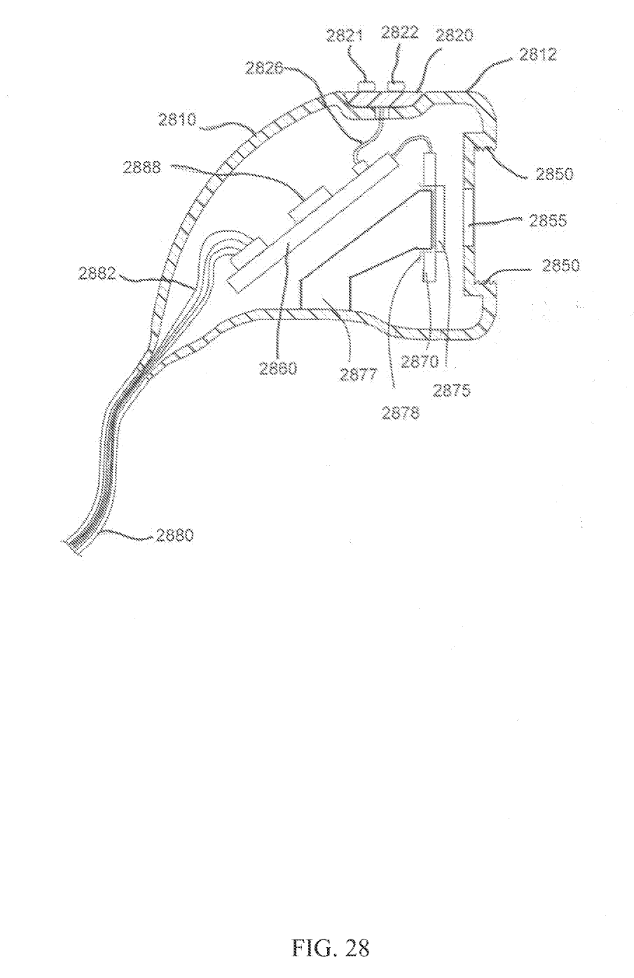

Illustrated in FIG. 28 is a cross-sectional view of an embodiment of an imaging device head 2812. The imaging device head 2812 may comprise a housing 2810 made of a suitably rigid material, such as plastic or metal. The housing 2810 may be sealed against fluids and gases so as to protect the internal circuitry and provide a suitable surface for sterilization and renewal. The imaging device head 2812 may further comprise a user input panel 2820 having buttons 2821 and 2822 for operation of the imaging device head 2812. Additional, buttons may be provided and the functionality of the buttons can be customized for a given procedure or a given operator. The control panel 2820 may be internally connected to other circuitry of the imaging device head 2812 by an electrical connector 2826.

As illustrated further in FIG. 28, imaging device head 2812 may comprise an optical mount system 2850, such as a C-mount system for receiving threaded accessories, for example one inch threaded accessories. A window 2855 may also be incorporated into the embodiment for facilitating the transmission of light from an optical accessory to an image sensor 2875. The image sensor 2875 may be mounted to a supporting printed circuit board or supportive substrate 2870. An electronic connector 2878 may be incorporated to electronically connect the image sensor 2875 to a main circuit or main printed circuit board 2860. A main wiring harness 2882 may be incorporated into a wired tether 2880 thereby electrically connecting the components of the imaging device head 2812 to a control unit.

The imaging device head 2812 may further comprise a memory 2888 or memory circuit allowing the storage of data within the imaging device head 2812. It will be appreciated that memory may be any data storage device that is capable of recording (storing) information (data). Data that may be stored or written into memory 2888 may include an identifying serial number that uniquely identifies an imaging device. Other data that may be stored or written into memory 2888 may include data such as the amount of the time the imaging device has been used, i.e., the hours of operation, or the amount of time the imaging device has been powered on. Data that may be written into memory 2888 may include sterilization data or renewal data, representing the working condition of the imaging device. Data that may be stored or written into memory 2888 may include data such as manufacturing date, date of last verification or quality control check, location of manufacture, i.e., may include name, city, state, street address and so forth, last control unit that the imaging device head was attached to, imaging device head diagnostic information, specific procedural settings for the imaging device head, or preferred settings for an operator or user, such as a surgeon. Data representing the above characteristics, or other indicia, of the imaging device may be recorded into memory within the imaging device.

The memory 2888 may be encryption protected so as to avoid tampering or unintended use and foreseeable misuse. It should be noted that a memory 2888 may be placed anywhere in the imaging device and not just the imaging device head without departing from the scope of the disclosure. The memory 2888 may comprise a permanent or semi-permanent portion allowing varying degrees of data durability. An embodiment may further comprise a heat sink 2877 for transferring heat away from circuitry and said image sensor 2875, and a thermal pad 2878 configured to be in physical contact with said image sensor 2875 and said circuitry and said heat sink 2877. The thermal pad 2878 may be configured to electronically isolate the image sensor 2875 from the patient and operator while enhancing the dissipation of heat generated by the image sensor 2875. As can be seen in the figure, the thermal pad 2878 may be disposed between the image sensor 2875 and may conform or be conformed to contact many surfaces of the heat sink 2877 or the image sensor 2875. The thermal pad 2878 may cover substantially all of the facing surfaces between the heat sink 2877 and image sensor 2875. It is within the scope of this disclosure to contemplate a multi piece thermal pad that may be place under the plurality of contact areas between the heat sink 2877 and the image sensor 2875. The thermal pad 2878 may also contact the image sensor 2875 at less than the entirety of the surface area of the image sensor. The thermal pad 2878 may be larger than the area of either the heat sink 2877 or the area of the image sensor 2875.

An embodiment of a single use imaging device for use with and communicating with a control unit may comprise a housing, an image sensor, an opening proximate to an optic mount that has been configured to facilitate the transmission of light from any attached optics to said image sensor. The imaging device may further comprise a memory for storing data representing any characteristics of the imaging device. In order to manage the heat created by the electronics in the imaging device the embodiment may further comprise a heat sink for transferring heat away from circuitry and said image sensor, and a thermal pad configured to be in physical contact with said image sensor and said circuitry and said heat sink. Wherein said thermal pad is further configured to electrically isolate said image sensor and said circuitry from said heat sink, and is further configured to thermally conduct heat generated by said circuitry and said image sensor. The thermal pad may be disposed between said image sensor and said heat sink such that image sensor is electrically isolated from said heat sink. A thermal pad may be configured to be in substantial contact with a single surface of said heat sink or maybe in substantial contact with a plurality of surfaces of said heat sink. In an embodiment the thermal pad may be in substantial contact with said heat sink across substantially all of a surface area defined by the heat sink surfaces facing said thermal pad, or at a portion that is less than the surface area defined by the heat sink surfaces facing said thermal pad. The thermal pad have a plurality of portions that are less than the surface area defined by the heat sink surfaces facing said thermal pad. The thermal pad may be of rigid configuration or may be flexible to conform to the surfaces of the image sensor and/or the heat sink

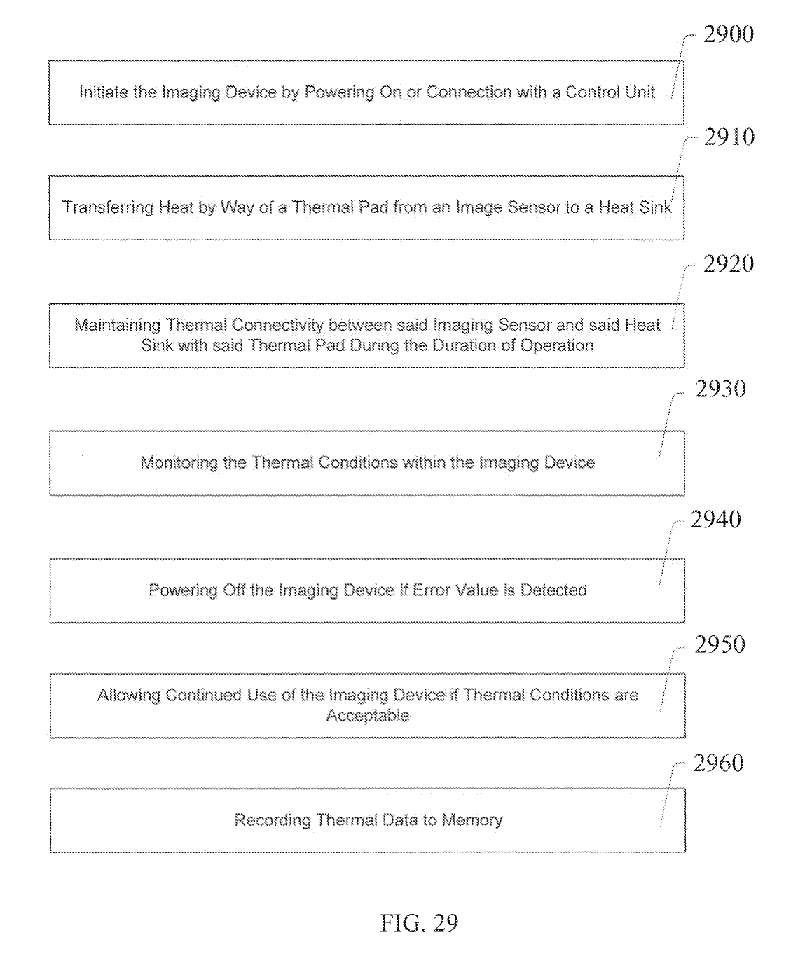

With primary reference to FIG. 29 a method for electrically isolating an image sensor within an imaging device while concurrently dissipating heat generated by the circuits therein. At 2900 the imaging device and system may be powered on for use. This may include the imaging device and system being initialized rather than actually being powered on. During operation electronic components, primarily the imaging sensor, will produce heat that may be dissipated. At 2910 heat is transferred from the imaging sensor to a heat sink via a thermal pad that may be optimized for transferring heat while insulating against electricity transmission. At 2920 the connection or contact of the imaging sensor and heat sink via a thermal pad is maintained. At 2930 the thermal condition of the imaging sensor and system may be monitored. Monitoring may be automatic with internal sensors and a control system, or monitoring may comprise observation by a user. At 2940 the system may be powered off if the monitoring returns a condition out side of predetermined range of parameters. The range of parameters may be electronic values or may comprise operational considerations such as resistance, inductance, perceived heat and the like. At 2950 the imaging device continues use until the procedure is finished. The imaging device may be powered down or place in a non-use state. Communication may be initiated with a control unit, wherein the data of the communication may comprise thermal related data. At 2960 any thermal related data my be recorded into memory on the imaging device. Such thermal data may be used to ensure quality control and operational fitness of the imaging device by preventing an over heated unit from continued use.

In the foregoing Detailed Description, various features of the disclosure are grouped together in a single embodiment for the purpose of streamlining the disclosure. This method of disclosure is not to be interpreted as reflecting an intention that the claimed disclosure requires more features than are expressly recited in each claim. Rather, as the disclosure reflects, inventive aspects lie in less than all features of a single foregoing disclosed embodiment.

It is to be understood that the above-described arrangements are only illustrative of the application of the principles of the disclosure. Numerous modifications and alternative arrangements may be devised by those skilled in the art without departing from the spirit and scope of the disclosure and the disclosure is intended to cover such modifications and arrangements. Thus, while the disclosure has been shown in the drawings and described above with particularity and detail, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A single use imaging device for use with and communicating with a control unit comprising:
a housing;
an image sensor;
an opening proximate to an optic mount and configured to facilitate transmission of light from said optics to said image sensor;
a memory comprising data representing characteristics of the imaging device;
a heat sink for transferring heat away from the image sensor;
a thermal pad that is in physical contact with said image sensor and said heat sink, such that said thermal pad electrically isolates said image sensor from said heat sink;
wherein said thermal pad is thermally conductive to conduct heat generated by said image sensor to said heat sink; and
an electronic communication circuit configured for providing electronic communication between said imaging device and said control unit.

2. The imaging device of claim 1, wherein said thermal pad is disposed between said image sensor and said heat sink, such that image sensor is electrically isolated from said heat sink.

3. The imaging device of claim 2, wherein said thermal pad is in substantial contact with said image sensor across substantially all of the surface area defined by a surface of the image sensor that faces the heat sink.

4. The imaging device of claim 2, wherein said thermal pad is in substantial contact with said image sensor at a portion that is less than all surface area defined by a surface of the image sensor that faces the heat sink.

5. The imaging device of claim 2, wherein said thermal pad is in substantial contact with said image sensor at a plurality of portions that are less than the entire surface area defined by a surface of the image sensor that faces the heat sink.

6. The imaging device of claim 1, wherein said thermal pad is in substantial contact with a single surface of said heat sink.

7. The imaging device of claim 1, wherein said thermal pad is in substantial contact with a plurality of surfaces of said heat sink.

8. The imaging device of claim 1, wherein said thermal pad is in substantial contact with said heat sink across substantially all surface area defined by the heat sink surfaces facing said thermal pad.

9. The imaging device of claim 1, wherein said thermal pad is in substantial contact with said heat sink at a portion that is less than the entire surface area defined by the heat sink surfaces facing said thermal pad.

10. The imaging device of claim 1, wherein said thermal pad is in substantial contact with said heat sink at a plurality of portions that are less than the entire surface area defined by the heat sink surfaces facing said thermal pad.

11. The imaging device of claim 1, wherein said thermal pad comprises a first portion that is thermally conductive and a second portion that is electrically isolating.

12. The imaging device of claim 1, wherein said thermal pad is flexible.

13. The imaging device of claim 1, wherein said thermal pad is substantially rigid.

14. The imaging device of claim 1, wherein said thermal pad is compressible.

15. The imaging device of claim 14, wherein said thermal pad is compressed between said image sensor and said heat sink.

16. A method for electrically isolating an image sensor in an imaging device while dissipating heat generated by said imaging sensor comprising;
powering on an imaging device comprising:
a housing;
an image sensor;
an opening proximate to an optic mount and configured to facilitate transmission of light from said optics to said image sensor;
a memory comprising data representing characteristics of the imaging device;
a heat sink for transferring heat away from the image sensor;
a thermal pad that is in physical contact with said image sensor and said heat sink, such that said thermal pad electrically isolates said image sensor from said heat sink;
wherein said thermal pad is thermally conductive to conduct heat generated by said image sensor to said heat sink; and
an electronic communication circuit configured for providing electronic communication between said imaging device and a control unit;
transferring heat generated by said imaging sensor to said heat sink via said thermal pad;
maintaining thermal connectivity between said imaging sensor and said heat sink via said thermal pad during the duration of operation; and
powering off said imaging device.

17. The method of claim 16, wherein the method further comprises monitoring thermal conditions of said imaging device; and wherein said imaging device is powered off when said monitoring of the thermal conditions returns a value out side of a predetermined range.

18. The method of claim 16, wherein the method further comprises monitoring thermal conditions of said imaging device; and wherein said imaging device remains powered on when said monitoring of the thermal conditions returns a value that is within a predetermined range.

19. The method of claim 16, wherein said imaging device transmits thermal data to the control unit.

20. The method of claim 19, wherein said control unit transmits an instruction to said imaging device in response to said thermal data.

21. The method of claim 16, wherein thermal data is recorded to said memory within said imaging device.

22. A system for obtaining imagery during a medical procedure comprising:
a single use imaging device comprising:
a housing;
an image sensor;
an opening proximate to an optic mount and configured to facilitate transmission of light from said optics to said image sensor;
a memory comprising data representing characteristics of the imaging device;
a heat sink for transferring heat away from said image sensor;
a thermal pad that is in physical contact with said image sensor and said heat sink, such that said thermal pad electrically isolates said image sensor from said heat sink;
wherein said thermal pad is thermally conductive to conduct heat generated by said image sensor to said heat sink; and
a control unit that electronically communicates with said imaging device.

23. The system of claim 22, wherein said thermal pad is disposed between said image sensor and said heat sink, such that image sensor is electrically isolated from said heat sink.

24. The system of claim 23, wherein said thermal pad is in substantial contact with said image sensor across substantially all of the surface area defined by a surface of the image sensor that faces the heat sink.

25. The system of claim 23, wherein said thermal pad is in substantial contact with said image sensor at a portion that is less than all surface area defined by a surface of the image sensor that faces the heat sink.

26. The system of claim 23, wherein said thermal pad is in substantial contact with said image sensor at a plurality of portions that are less than the entire surface area defined by a surface of the image sensor that faces the heat sink.

27. The system of claim 22, wherein said thermal pad is in substantial contact with a single surface of said heat sink.

28. The system of claim 22, wherein said thermal pad is in substantial contact with a plurality of surfaces of said heat sink.

29. The system of claim 22, wherein said thermal pad is in substantial contact with said heat sink across substantially all surface area defined by the heat sink surfaces facing said thermal pad.

30. The system of claim 22, wherein said thermal pad is in substantial contact with said heat sink at a portion that is less than the entire surface area defined by the heat sink surfaces facing said thermal pad.

31. The system of claim 22, wherein said thermal pad is in substantial contact with said heat sink at a plurality of portions that are less than the entire surface area defined by the heat sink surfaces facing said thermal pad.

32. The system of claim 22, wherein said thermal pad comprises a first portion that is thermally conductive and a second portion that is electrically isolating.

33. The system of claim 22, wherein said thermal pad is flexible.

34. The system of claim 22, wherein said thermal pad is substantially rigid.

35. The system of claim 22, wherein said thermal pad is compressible.

36. The system of claim 35, wherein said thermal pad is compressed between said image sensor and said heat sink.
